(19) United States
(12) Patent Application Publication
(10) Pub. No.: US 2026/0321367 A1
(43) Pub. Date: Oct. 15, 2026

(54) USING ELECTROPHORESIS FOR DISEASE DETECTION BASED ON CONTROLLED MOLECULAR CHARGE

(71) Applicant: HEMEX HEALTH, INC., PORTLAND, OR (US)

(72) Inventors: Peter Galen, Portland, OR (US); Arwa Fraiwan, Cleveland, OH (US); John Hinshaw, Portland, OR (US); Umut Atakan Gurkan, Shaker Heights, OH (US)

(21) Appl. No.: 19/478,305

(22) PCT Filed: Apr. 29, 2024
(86) PCT No.: PCT/US2024/026884

§ 371 Date: Oct. 24, 2025

Related U.S. Application Data
(60) Provisional application No. 63/498,768, filed on Apr. 27, 2023.

(51) Int. Cl. G16H 10/40; G16H 50/20

(52) U.S. Cl. CPC G16H 10/40; G16H 50/20

(57) ABSTRACT

Electrophoresis identifies presence of hemoglobin A1c (HbA1c) in a patient sample based on one or both of a charge state and a mass of a HbA1c complex, which is HbA1c conjugated to a binder having a controlled charge and/or mass. The controlled charge and/or mass of the HbA1c complex generates a controlled migration velocity and/or migration pattern over a period of time of the HbA1c complex when an electric potential is applied to a substrate on which the HbA1c complex is applied. The controlled migration velocity and/or migration pattern can be adjusted to separate the HbA1c complex band from other hemoglobin bands, markers, and/or other compounds. The disclosed systems and methods identify presence of and optionally quantification of HbA1c based on the HbA1c complex band and one or both of its migration pattern and the migration velocity.

### CROSS-REFERENCE TO RELATED APPLICATIONS

This application is a 371 filing of international application PCT/US24/26884, filed Apr. 29, 2024, which claims priority and benefit from U.S. Provisional Patent Application No. 63/498,768, filed Apr. 27, 2023, titled, “HbA1c point-of-care test leveraging immunodetection and microchip electrophoresis,” which is incorporated herein by reference in its entirety for all purposes.

This application is also related to U.S. application Ser. No. 17/504,363, filed Mar. 27, 2024, titled “USING ELECTROPHORESIS FOR DISEASE DETECTION BASED ON CONTROLLED MOLECULAR CHARGE,” which is a continuation of U.S. application Ser. No. 17/504,363, filed Oct. 18, 2021, which is a continuation of U.S. application Ser. No. 17/232,130, filed Apr. 15, 2021, now U.S. Pat. No. 11,255,815, which claims priority and benefit from the U.S. Provisional Patent Application 63/010,570, filed Apr. 15, 2020 and titled, “INFECTIOUS DISEASE DETECTION & DIAGNOSTICS,” which are all incorporated herein by reference in their entirety for all purposes.

This application is also related to PCT Application No. PCT/US2021/027566, filed Apr. 15, 2021, titled “USING ELECTROPHORESIS FOR DISEASE DETECTION BASED ON CONTROLLED MOLECULAR CHARGE,” which is incorporated herein by reference in its entirety for all purposes.

### BACKGROUND

Electrophoresis and immunoassays are both powerful tools used to test for many disease conditions. These include infectious disease, chronic disease, measurement of metabolic condition and many more. The tests to diagnose and manage these diseases are generally expensive, slow, and complex and require a central laboratory, or if used in a remote location, may not offer the sensitivity and performance required for an accurate determination of disease status. Disease diagnostics are critical to global health in all countries—low, middle, and high income countries alike—to prevent community spread of infectious disease and undiagnosed chronic disease and to avoid unnecessary morbidities and mortalities. Oftentimes, disease diagnostics need a requisite concentration of disease agent or biomarker—such as a protein associated with, produced by, or produced in response to a virus or a sugar complex on a biomarker—in a patient's sample before the diagnostic tool can detect the disease. Unfortunately, infected patients, for example, are required to wait a period of time after a suspected or confirmed exposure to the disease to allow for the diagnostic tool to give a result with clinically acceptable sensitivity and specificity. Symptomatic patients may experience delays in effective treatment during this wait period because medical personnel are unable to confirm a diagnosis with enough statistical certainty until the wait period expires and the disease condition is confirmed by the diagnostic test. People may be unaware of their infection during this wait period, which results in unintentional community spread of the disease including to other people who may be more medically complicated than the originally infected person. This wait period for conventional infectious disease diagnostics can last several days to a week or sometimes longer and is dependent upon the concentration of the disease biomarker in the patient sample that can be detected with the available diagnostic tools.

Patients suffering from chronic disease likewise suffer from insufficient diagnostic testing that is expensive and does not have the desired sensitivity and specificity. Oftentimes, these chronic patients suffer high rates of morbidity and mortality because they remain undiagnosed due to lack of affordable and available testing.

Conventional disease diagnostics with the highest sensitivity and specificity require laboratories to process the patient samples, which are expensive resources not widely available in underserved regions. These laboratories can also become overwhelmed during periods of higher rates of infectious disease in a community in low, middle, and high income countries alike because performing their disease testing techniques often requires substantial time and expensive equipment. For example, patients presumed to be infected with the novel coronavirus of 2020—severe acute respiratory syndrome coronavirus 2 (SARS-CoV-2)—that causes coronavirus disease (COVID-19) typically give a nasal-pharyngeal or oropharyngeal sample that is tested in a central laboratory using a highly sensitive reverse transcriptase polymerase chain reaction (RT-PCR) technique in which the ribonucleic acids (RNA) or other nucleic acid of the virus are isolated for detection and, in some cases, amplified to confirm an infection.

The RT-PCR diagnostics are powerful tools; however, they also require sophisticated laboratory equipment to isolate and amplify the RNA and skilled laboratory technicians to perform analysis of the results. Further, the laboratory equipment that isolates and amplifies the nucleic acid segments of the target virus is expensive. Even in developed countries like the United States, community need for such equipment is often driven based on typical infectious disease testing needs, rather than pandemic or epidemic conditions. Scaling infectious disease diagnostics laboratories along with skilled personnel and sophisticated equipment to meet greater than normal testing needs is an expensive and time-consuming process that often trails the need so greatly to make its return on the investment in resources poor or nearly non-existent. High income countries are slow to respond to this scaling need for many reasons including a lack of resources and infrastructure. Sadly, middle and low income countries may not be able to respond to this scaling need at all because the infrastructure never existed or due to a severe lack of resources. A similar effect happens with chronic disease especially in underserved areas. Often the tests are too expensive or require infrastructure that does not support transporting samples to a remote central laboratory or effectively communicating results.

In general, global health is increasingly in need of highly sensitive-highly specific point-of-care (POC) diagnostics for patients to deliver faster, more accurate results close to the patient. One type of POC diagnostic device is a rapid diagnostic test (RDT) that is designed for low-cost, mass scale deployment to clinical and non-clinical settings. Faster, more accurate results allow sick patients to receive treatment—sometimes life-saving treatment—sooner, which allows community leaders to set informed healthcare and economic policies, and gives healthcare personnel much needed diagnostic infrastructure support to diagnose and treat ill patients. These POC diagnostics improve community health and safety and avoid overwhelming critical care treatment facilities so that the most severely infected or ill patients can easily access treatment. Early diagnosis helps reduce the risk of untreated infection or underlying chronic condition becoming severe and of unnecessary community spread of myriad diseases ranging from infections like malaria, measles, and SARS-CoV-2, to chronic diseases like cancer, coronary artery disease, and thyroid disease. Also, POC diagnostics need to be easy to operate and do not require highly skilled technicians to determine a diagnostic result. Still further, POC diagnostics tend to be much less expensive than laboratory tests, which makes their deployment in developing countries and on mass scales in developed countries realistic and effective.

Currently, available POC diagnostics for many diseases—including COVID-19—often suffer from a low sensitivity and specificity, which, for example, makes it difficult to discriminate patients with a confirmed infection from patients with a confirmed healthy or non-infectious sample. Many POC diagnostics detect antigens, antibodies, or biomarkers associated with a health condition—such as SARS-CoV-2 virus nucleocapsid protein for COVID-19 or histidine-rich protein-2 (HRP2) for malaria—using lateral flow assays that require relatively high concentrations of the compound to be present in the patient sample. These tests can give inconclusive results (e.g., a band that is very faint or is not detectable by available equipment or personnel, which make a visual determination of the results difficult or impossible), or simply are performed incorrectly due to lack of trained personnel. They also rarely provide a quantitative result—the actual concentration of the biomarker of concern, which can be critical to clinical decision-making.

Further, diabetes is one of the most important health issues in the world. The number of people living with diabetes has quadrupled since 1980 with the greatest increase in prevalence in low and middle-income countries. Not only is diabetes a major cause of morbidity and mortality, but it can also devastate the finances of families and strain countries' financial resources. The World Health Organization (WHO) is currently challenging governments to achieve a 25% reduction in deaths due to diabetes and other non-communicable diseases by 2025. A key part of the WHO program calls for improved diagnosis and management of diabetes in primary healthcare. For many years, the glycated hemoglobin HbA1c test has been used as a reliable biomarker for diabetes and prediabetes, since it is the best indicator of glucose control over time. Due to the relative ease of testing of HbA1c versus the fasting plasma glucose test, it is often the preferred option in developing countries.

Current HbA1c tests, however, may be inaccurate due to hemoglobinopathies, which are also prevalent in developing countries, and availability has been limited by the lack of affordable point-of-care (POC) solutions. Although regular monitoring of HbA1c in diabetics improves patient outcomes, current testing options, if available at all, are: (a) expensive laboratory tests with delayed patient feedback, (b) expensive POC options not suitable for remote areas, or (c) expensive and hard-to-use home testing kits. There is a significant need for an affordable, portable, easy-to-use, POC test that rapidly quantifies HbA1c, which would enable widespread screening and allow better monitoring and management of treatment.

All these drawbacks limit the use and effectiveness of POC diagnostics for many diseases, especially those where the concentrations of the compound being tested are low or other factors like parallel disease states, such as hemoglobinopathies, are present and can obfuscate the results—making the process of obtaining accurate results unreliable, undetectable, or simply are too expensive or delay treatment.

The limitations of the POC diagnostics for infectious diseases like COVID-19 can lead to unintentional disease spread, waste of valuable healthcare resources, and unnecessary isolation of healthy patients presumed infected due to a false-positive test result, among many other immediate and downstream health, safety, and economic consequences. The limitations of POC diagnostics for non-infectious diseases like diabetes can lead to failure to diagnose and treat patients before disease progression causes morbidities or mortalities.

Therefore, the healthcare industry needs improved infectious and non-infectious disease diagnostics that are highly-sensitive, highly-specific, fast, affordable, easy to use, and deployable on a mass scale all over the world.

### SUMMARY

Disclosed methods and systems identify the presence of a compound, such as hemoglobin A1c (HbA1c) in a patient sample using electrophoresis. The presence of the compound is based on a charge and/or mass profile of a binder that binds to a target compound to create a bound complex with a controlled compound charge state and/or mass. The controlled compound charge and/or mass state is the total net charge and/or total mass of the compound bound or conjugated to the binder. The binder has a controlled binder charge and/or mass profile that, when bound with the known charge and mass of the target compound, creates the concentration of a bound complex having a controlled compound charge and mass. The bound complex and remaining unbound binder are then applied to a substrate. In some examples, the substrate is an electrophoresis substrate to which an electric potential is applied for an interval. Bound complex and unbound binder are attracted to the electrode of the opposite charge and migrate across the substrate at a constant or varying migration velocity over the interval when the electric potential is applied. The different migration velocities of the bound complex, other compounds in the patient sample, and possibly unbound binder at their migration velocities create a migration pattern on the substrate over the interval. The migration pattern is the manner in which the bound complex behaves as it migrates across the substrate, including but not limited to when it separates from other compounds in the applied patient sample, how it migrates, its band width, when it increases or decreases velocity, and the like. The migration of the bound complex and unbound binder over time at the migration velocity ultimately results in bound complex and unbound binder band(s) (if present) during or at the end of the interval. The migration pattern dynamically changes throughout the interval as components on the substrate—including the bound binder-target compound complex—migrate in response to the applied electric potential. The presence of the compound is identified based on the band(s) and one or both of the migration pattern and the migration velocity. The migration pattern on which the presence of the compound is determined can be captured using any one or more optical technique(s), such as image capture(s), continuous imaging segment(s), video, density analysis (evaluating light transmission), and the like during or at the end of the interval.

In some embodiments, the binder or the target compound is optically detectable or visible during and after an electrophoresis immunoassay run. The binder and the target compound can both be optically detectable or visible during and after the electrophoresis immunoassay run. Optically detectable or visible means that the band that forms as the bound or conjugated compound complex migrates across the electrophoresis substrate is able to be visibly detected. Visible detection could be that it is visible to a human eye without an aid or with any suitable visibility aid. For example, hemoglobin (Hb) is visible to a human eye without a visibility aid. However, visibility of Hb is enhanced when the Hb band is excited by an emission source, such as a light source emitting light within a range of 400-460, where hemoglobin absorption is highest. For example, Hb has absorption peaks at a wavelength of about 414 nm, which can be optically captured in an optical image, video, or other optical capture when using a camera, densitometer, or other optical device that measures or captures an optical feature or characteristic of the substrate before, during, and/or after the immunoassay run, such as band spatial intensity that measure the progression of the intensity of the band from its leading edge to its center and to its trailing edge. The fluorescing Hb band creates a clear data point in the optical capture to then evaluate. The evaluation can include determining the velocity, intensity, density, or the like to analyze whether Hb or one of it sub-types is present in the patient sample under evaluation.

Alternatively, in other embodiments, the binder or the target compound can have a label that is visible, fluorescent, or otherwise optically detectable. Without a label or staining binder, some target compounds or bound complexes are not optically detectable at all. Use of a label with the binder or target compound enhances the ability to detect them without the need for staining and washing normally used in electrophoresis immunoassays. In addition, a fluorescently labeled bound complex is detectable at much smaller concentrations than with standard staining. The label is added to either the binder and/or the target compound before the binder conjugates to the target compound. The labeling of the binder or compound creates a labeled bound binder-target complex. Specifically, the labeled bound complex having a controlled charge and mass profile allows for optically detectable bands to form in the migration pattern and, in some examples, during the active assay run. Labels are used when the compound is difficult to optically detect, such as when the volume of the compound and/or the binder is too low to otherwise optically detect it. However, labels, and fluorescent labels, can also be used with binders and/or compounds that are visible, whether easily visible or more difficult to optically detect, to enhance or alter the visibility of the binder and/or the target compound.

### BRIEF DESCRIPTION OF THE DRAWINGS

Non-limiting and non-exhaustive embodiments of the invention are described with reference to the following drawings. In the drawings, like reference numerals refer to like parts throughout the various figures, unless otherwise specified, wherein:

FIG. 1 shows an example diagnostic system that separates Hb protein types using a binder with a controlled charge and/or mass.

FIGS. 2A and 2B show examples of methods of identifying hemoglobin A1c (HbA1c ) in a patient sample using the disclosed electrophoresis systems and methods.

FIG. 3 shows an example method of curating a binder having a controlled charge and/or mass and conjugating the binder to a target compound.

FIGS. 4A and 4B shows example substrate with a prepared patient sample applied to it and binders without and with a label, respectively, according to aspects of the disclosure.

FIGS. 5A and 5B show an example patient sample and patient sample preparation with a target compound or biomarker and a binder with a label.

FIGS. 5C and 5D show an example patient sample and patient sample preparation with a target compound or biomarker and a binder without a label.

FIGS. 6A and 6B show an example patient sample and patient sample preparation without a target compound or biomarker and a binder with a label.

FIGS. 6C and 6D show an example patient sample and patient sample preparation without a target compound or biomarker and a binder without a label.

FIG. 7 shows a graph of example unbound binder and three different peak charge states of bound binder-target complex.

FIGS. 8A and 8B show an example migration pattern on a substrate without a bound complex band and with a bound complex band, respectively, after an assay run is complete, according to aspects of the disclosure.

FIGS. 9A and 9B show an example migration pattern on a substrate for Hb separation into sub-types with a binder without a label conjugated to HbA1c.

FIGS. 10A and 10B show an example diagnostic system with an optical imager and substrates with bound complex before and during or after the assay run, respectively, is complete.

FIG. 11 shows an example patient sample cartridge with an integrated substrate that shows a migration pattern of a bound complex.

FIG. 12 is an example diagnostics system, according to aspects of the disclosure.

FIGS. 13A and 13B are process flow diagrams of methods to identify presence of a compound in a patient sample, according to aspects of the disclosure.

FIGS. 14A and 14B show examples of migration patterns of two patient samples having different concentrations of bound complex on a substrate, respectively.

FIG. 15 shows an example process flow diagram of analyzing band intensity of unbound binder and bound complex in a migration pattern to determine a concentration of the compound.

### DETAILED DESCRIPTION

The subject matter of embodiments disclosed herein is described with specificity to meet statutory requirements and to convey the scope of the subject matter to those skilled in the art, but this description does not limit the scope of the claims. The disclosed subject matter may be embodied in other ways, may include different elements or steps in the same or a different order, and may be used in conjunction with other existing or future technologies.

The disclosed diagnostic methods and systems are able to provide highly-sensitive, highly-specific test results using a patient sample with a low concentration of target compound and/or patient samples that have multiple, difficult to separate or easily obfuscated compounds. The test focus can be an infectious or chronic disease, metabolic state, or a pre-disease condition. A target compound is a compound or biomarker of interest for which a patient sample is being tested. The target compound in immunoassays, for example, is often an indicator or biomarker—antigen, antibody, byproduct, or otherwise—of an infection, disease state, or pre-condition in a patient. The target compound is the measurable component of the patient sample that can determine whether or not the patient is infected with the disease, has a pre-disease condition, or is experiencing a metabolic state of interest (e.g., pregnancy or ingestion of a compound like a performance enhancing substance). The patient sample can be any suitable tissue or fluid sample from the patient. For example, most commonly in POC diagnostics, the patient sample is blood, nasal-pharyngeal or oropharyngeal fluids, urine, saliva, mucus, tissue cells, and the like. A patient can be human or animal. The patient sample can also be a plant sample or other non-biologic sample as well.

The disclosed methods and systems can take advantage of existing POC electrophoresis systems, such as the Gazelle® platform commercially available from Hemex Health®, Inc. or other POC electrophoresis devices. Many conventional immunoassays do not consider charge or mass of the binder, the compound, or the bound complex as part of the analysis because charge is not relevant to the migration of those compounds on the assay. This is especially true when optically detectable binders, compounds, complexes (binders conjugated to compounds), and/or labels are included. For example, a lateral flow assay is based on flow of a patient sample along a substrate and over one or more control lines to which some target compound or biomarker would bind if it is present in the patient sample. Lateral flow assays do not rely on an applied electric potential to generate the migration of any compounds so whether a molecule has a total net charge or is in a particular charge state is not relevant to the compound's ability to migrate across the substrate and bind with test lines. Further, mass of the binder is irrelevant in lateral flow assays because speed of diffusion is irrelevant to the testing technique. Throughout this disclosure, reference to the target compound includes any compound, molecule, biomarker, or other indicator of disease, pre-disease condition, or metabolic state.

In non-infectious diseases and metabolic states, the target compound or antigen available for detection is sometimes present in the patient sample at a volume that is visible, either by an unaided human eye or with a visibility aid, such as light emitted at a specific wavelength or within a specific wavelength range to cause excitation of the binder and/or target compound, which results in it fluorescing at an expected wavelength. For example, Hb is a type of protein with various sub-types, each of which can be used to evaluate one or more disease states and/or metabolic conditions of a patient. During a first or beginning portion of the assay run, total Hb is visible as a band on an electrophoresis substrate when an electric potential is applied. During a second or latter portion of the assay run, the total Hb band separates into its various sub-types. Typically, a calibrator is also included on that substrate or within a mixing chamber or receiving chamber of the patient sample. For example, the calibrator is stored or added to a cartridge, such as a microchip electrophoresis cartridge, which includes a substrate and electrodes on opposing ends of the substrate. A calibrator is a compound having a known electric charge, mass, migration velocity, and migration pattern. Because of its known properties, it will have a known or predetermined behavior throughout the assay run, and therefore can be used as a relative point against which to compare the behavior of the total Hb or any of the Hb sub-types, such as HbA1c. Using a calibrator eliminates the need to calibrate the system as frequently because relative values and behaviors, rather than absolute values and behaviors, of the total Hb and its sub-types are analyzed with the calibrator values and behaviors.

In an example, the substrate is a cellulose acetate paper housed in the microchip. The patient sample is blood, in some examples, which is prepared for the immunoassay run by lysing it in a diluent with the calibrator. In some examples, a lysing agent, such as saponin, is also used with the diluent to prepare the patient sample. A binder having a controlled molecular charge and/or controlled molecular weight, such as a monoclonal HbA1c antibody, is added to the prepared sample, which is then allowed to incubate. In an example, the prepared patient sample incubates in a nutating shaker for 30-45 minutes. Additional steps or alternative incubating or patient sample preparation techniques can be employed. Once prepared, the patient sample is applied to the microchip substrate, which is cellulose acetate in this example using a buffer. In an example, the buffer is Tris-Borate EDTA at a pH of ~8.4. The assay run begins when a voltage of 250 V is applied across the electrodes causing the migration of the compounds. In this example, the voltage of 250 V is applied for 8 minutes although other example assay runs could apply the voltage for longer or shorter periods of time and can apply the voltage in one or multiple time segments. As the calibrator and the conjugated binder-HbA1c compound, among other Hb sub-types migrate across the substrate.

In this example, the system captures live images and/or video recording of the immunoassay run during, throughout, and/or at the end of the run. The images and/or video are captured under both white light and UV illumination at a wavelength of 410 nm in this example. Other embodiments employ either white light or light within a specific wavelength range. Still other embodiments employ light within a specific wavelength range alone. These wavelengths are intended to enhance the visibility of the bands produced by their respective migrating compounds. The wavelength is chosen based on having the greatest absorption for one or more of the migrating compound(s), binder, and/or calibrator. Here, the wavelength of 410 nm is chosen to take advantage of the peak optical absorption of Hb, which occurs at 414 nm. It also allows for optical detection of Hb bands that are more difficult to see or separate from total Hb, such as HbA1c bands. HbA1c bands are not easily visible under white light and are sometimes in low concentrations in the patient sample, which make them difficult or impossible to detect without visualizing with light near 414 nm. Often, under white light, the limit of detection for HbA 1c bands is around 10% while the limit of detection under illumination with light at 410 nm is <2%.

In an example, the disclosed systems can include a point-of-care (POC) microchip electrophoresis system for hemoglobin (Hb) variant (sub-type) testing. Such a system can use a single-or multi-use cartridge based diagnostic platform that separates Hb protein sub-types present in a patient's blood sample. The patient blood sample is applied on a piece of cellulose acetate paper, in this example, that is housed in a micro-engineered chip with a controlled environment and electric field. Separating total Hb in a patient sample from its sub-types can be divided into two steps based on processing time. Total Hb (the combined compound of HbA0 and its sub-types along with other Hb types that may be present) separates from the calibrator during a first period of time of the assay run. During a second period of time of the assay run, Hb sub-types separate from each other, such as HbA0 separating from HbA2, if present, and Hb A1c. Other sub-types of Hb can separate from total Hb as well, including HbS, if present, other sub-types of HbA, HbC, and HbE. These latter sub-types of Hb are typically used to diagnose various hemoglobinopathies, including sickle cell disease and beta thalassemia.

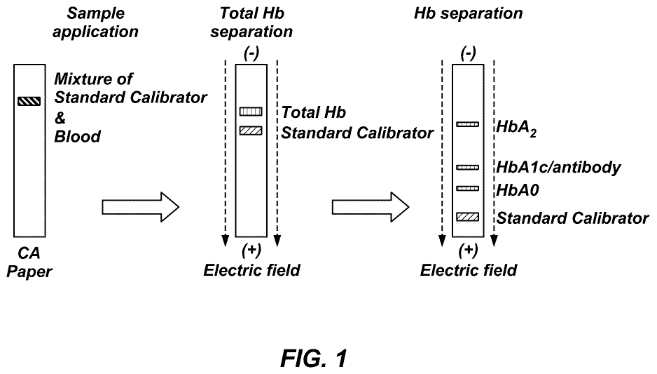

FIG. 1 shows an example immunoassay system 100 that separates total Hb from the calibrator. Here, a mixture of the calibrator and patient blood sample is applied to an electrophoresis substrate 102, which is cellulose acetate in this example. The electric field is applied in a first step or interval of the immunoassay run 104, which causes the total Hb (that includes an HbA1c complex of conjugated binder to HbA1c present in the patient sample) to separate from the standard calibrator. In this example, the first step is to separate all types of Hb protein (total Hb) from the incorporated Hb level measurement standard calibrator, which is a customized fiducial marker here. The calibrator has a greater negative charge state and/or a smaller mass than the total Hb, which causes it to migrate towards the positive electrode more quickly that the total Hb. Because it is either or both of more negatively charged and lighter in mass than the total Hb, it has a correlating faster migration velocity. The calibrator also has a known migration pattern because of its known charge state and mass, which is used to evaluate the relative movement of the total Hb and the sub-types of Hb as they separate from the total Hb compound band. As the calibrator and the total Hb continues to migrate, the calibrator remains the compound band that has the highest migration velocity while the total Hb sub-types begin to separate based on their respective charges and mass. Alternatively, the calibrator could be designed to have a migration velocity that is slower than any one or more of the Hb types or sub-types, as needed.

The second step or interval of the immunoassay 106 occurs when there are different Hb types in the patient sample. Variations in Hb mobilities for different Hb sub-types allow separation to occur along the cellulose acetate paper. The variations in Hb mobilities for each of the different Hb sub-types can be controlled using a binder, such as the HbA1c antibody, shown in FIG. 1. HbA1c has a specific, single binding site for the curated HbA1c antibody, in an example, and a known, controlled mass. Alternatively, HbA1c could have multiple binding sites for another curate HbA1c antibody in another example. Each molecule of HbA1c also has a known charge and mass, which when the HbA1c antibody conjugates to the HbA1c, produces a bound HbA1c complex with a controlled charge and mass.

Referring again to FIG. 1, the controlled charge state and mass of the bound HbA1c complex differs from the charge and mass of HbA0 and HbA2, which are also present in the patient blood sample shown in FIG. 1. Here, HbA0 has a greater negative charge and/or lower mass than the bound HbA1c complex, and the bound HbA1c complex has a greater negative charge and/or lower mass than the HbA2 compound, which is reflected in its migration velocity and migration pattern (distance, location, etc.) over time throughout the immunoassay run. As shown in FIG. 1, the total Hb compound has a band width approximately equal to the band width that is thicker than the respective band widths of each of its sub-types as they separate from each other during the second step 106. Controlling the charge and mass of the HbA1c protein by conjugating a binder to it changes its electrophoretic mobility and allows it to be separately measured from HbA0 (non-glycated HbA0).

The cartridges shown in FIG. 1 can be inserted into a reader, such as an automated reader that receives the cartridge, applies the voltage to the cartridge electrodes, and images the cartridge before, during and/or after the immunoassay run. This reader can also evaluate the captured images and/or video itself on an integrated processor or it can transmit the images to a remote processor for evaluation. The images, whether analyzed using an on-device or remote processor(s), include data about the bound HbA1c complex and its migration velocity and/or migration pattern throughout the immunoassay run. This data can include band data, such as the bands that form when the compounds migrate across the substrate. Each band that appears in one or more of the captured image(s) or video of the assay run corresponds to one compound or a complex of compounds. For example, total Hb in the first interval of the assay run shown in FIG. 1 corresponds to a complex of HbA0, HbA1c conjugated to an HbA1c antibody, and HbA2.

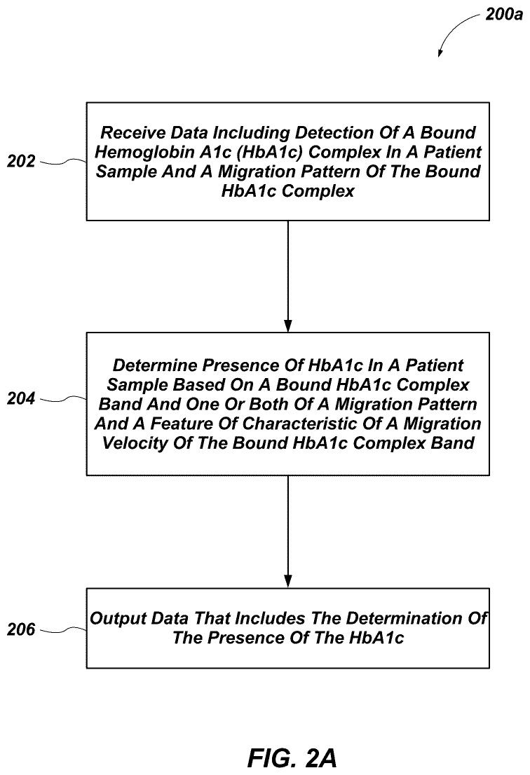

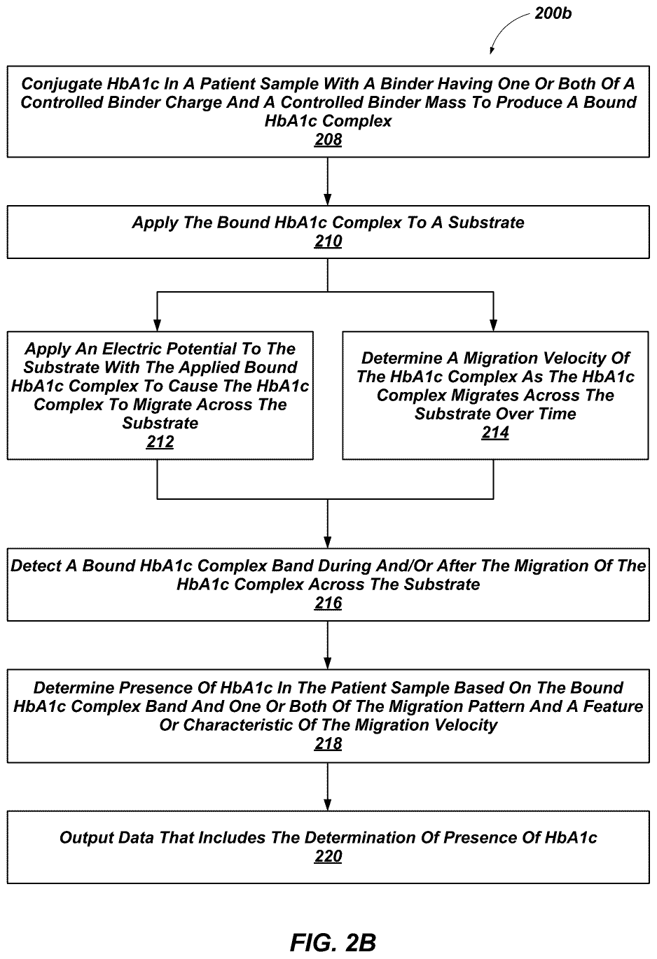

FIG. 2A shows a method of identifying hemoglobin A1c (HbA1c ) in a patient sample 200a using the disclosed electrophoresis systems and methods. The method can include processing steps that occur in an on-device or remote processor(s) of a system that includes a reader that receives a cartridge with the patient sample and performs the immunoassay run by applying electric potential. Here, data is received that includes detection of a bound HbA1c complex in a patient sample and a migration pattern of the bound HbA1c complex through the immunoassay run 202. Such data can be received, for example, at a processor for evaluation and can include images or video of the cartridge on which the immunoassay run occurred or is actively occurring. The disclosed systems and methods determine presence of the HbA1c in the patient sample based on the bound HbA1c complex band and one or both of the migration pattern and a feature or characteristic of the migration velocity of the bound HbA1c complex band 204. The determination can occur as a processing step in an on-device and/or remote processor or computing system or device. Presence of the HbA1c in the patient sample can be based on a visible or optically detectable technique to identify bound HbA1c complex on the electrophoresis substrate.

In some examples, the presence of HbA1c is determined by illuminating a bound HbA1c complex band that appears as the bound HbA1c complex migrates across the substrate when an electric potential is applied. The migration occurs because the bound HbA1c complex has a controlled charge and/or controlled mass that is drawn at a migration velocity and produces a migration pattern across the substrate when the electric potential is applied. The migration velocity is the velocity at which the bound HbA1c complex migrates across the substrate, which is determined by either controlled charge (with an insignificant mass relative to the total mass of the HbA1c complex) and/or controlled mass that impacts the migration velocity of the HbA1c complex as it migrates across the substrate. The charge of the bound HbA1c complex is controlled by the binder having a controlled charge and binding to a known single or multiple binding sites on the HbA1c compound. The HbA1c compound has a known charge without the conjugated binder. When the binder has a controlled charge, its charge can be added to the known charge of the HbA1c compound. If the binder binds at only a single binding site on the target compound, such as with HbA1c, a total net charge of the bound HbA1c complex is equal to the charge of the HbA1c compound plus the charge of one binder molecule. If the binder binds to multiple binding sites on the HbA1c compound, a total net charge of the bound HbA1c complex is equal to the charge of the HbA1c compound plus the charge of the binder molecule multiplied by the number of binding sites to which it is conjugated on the HbA1c. In one example, the controlled charge of the binder is selected to intentionally increase or decrease the charge of the bound HbA1c complex to increase or decrease its migration velocity across the substrate. In another example, the controlled charge of the binder is selected to be neutral or a net “0” to intentionally avoid changing the charge state of the HbA1c. A binder with a neutral or net 0 charge will produce a bound HbA1c complex that has the same charge state as the HbA1c compound by itself.

As mentioned above, the migration velocity of the HbA1c complex can also be determined by the controlled mass of the binder that conjugates to the HbA1c compound. The binder has a controlled mass, and the HbA1c compound has a known mass. Together, the total mass of the bound HbA1c complex is the controlled mass of the binder added to the known mass of the HbA1c compound. In some circumstances, the controlled mass of the binder is negligible or relatively small compared to the known mass of the HbA1c compound. Such binders with negligible or relatively small mass compared to the HbA1c compound do not change the migration velocity of the bound HbA1c complex within a margin of error. In other examples, the controlled mass of the binder is high enough and/or has enough mass relative to the total mass of the bound HbA1c complex to change the migration velocity of the bound HbA1c complex across the substrate. When the controlled binder mass is high enough to affect the migration velocity of the bound HbA1c complex, it slows down the migration velocity of the bound HbA1c complex compared the migration velocity of the HbA1c compound by itself.

In some examples, the binder has a neutral charge that does not change the migration velocity of the bound HbA1c complex, and a mass that is high enough to slow the migration velocity of the bound HbA1c complex. In another example, the binder has a negative charge that increases the total net (negative) charge of the HbA1c compound alone, and a negligible mass that does not change the total mass of the HbA1c compound in a meaningful way to affect the migration velocity of the bound HbA1c complex. In yet another example, the binder has both a negative charge and a mass that changes the total mass of the HbA1c compound in a way that increases the migration velocity of the bound HbA1c complex resulting in either faster, slower, or the same migration velocity. Essentially, either the controlled charge or the controlled mass of the binder or both the controlled charge and controlled mass of the binder can change the migration velocity of the HbA1c compound when the binder is conjugated.

Further, in one example, the HbA1c compound has a single binding site for the curated HbA1c antibody, which affects the total net charge and total mass of the bound HbA1c complex. The bound HbA1c complex has a corresponding single total charge state and total mass, which results in a corresponding single charge state and single total mass for the conjugated binder to the HbA1c compound. However, in another example, the HbA1c compound has multiple binding sites for the same or a different curated HbA1c antibody or other binder. In the multiple binding site example, the bound HbA1c complex can have multiple total charge states and/or multiple total mass depending on the number of binder molecules conjugated to the HbA1c compound. For example, if the HbA1c has 3 binding sites, the resulting bound HbA1c complex will have three total charge and/or total mass states, depending on whether the binder has a controlled charge that changes the total charge state(s) of the bound HbA1c complex from a charge state of the HbA1c compound alone and/or whether the binder has a controlled mass that changes the total mass of the bound HbA1c enough to affect its migration velocity.

Referring again to FIG. 2A, the disclosed systems and methods can output the data that includes the determination of the presence of the HbA1c 206. The output can be a data packet output from a processor, software module, or algorithm. Alternatively or additionally, the output can be a data packet that is transmitted to another computing element, device, or system, such as a data packet that is transmitted to a user device or remote server for further processing. In one example, the data can be transmitted to a display on a user device, whether that display is integrated within the same device that houses the processing or processing circuitry or is a component of a remote user device. Alternatively, the output can be transmitted to another processor for further data processing rather than displayed.

FIG. 2B shows another example method of identifying hemoglobin A1c (HbA1c ) in a patient sample 200b using the disclosed electrophoresis systems and methods. Here, the HbA1c in a patient sample is conjugated with a binder having one or both of a controlled charge and/or a controlled mass to produce a bound HbA1c complex 208. The conjugation of the binder and the HbA1c can occur in a sample mixing chamber within or external to any of the disclosed systems. In some examples, the conjugated occurs in a sample mixing chamber that is applied to a substrate of a microchip cartridge that is inserted into a reader that applies the electric potential to the substrate, images the substrate during and at the end of the assay run, and processes some or all of the data to determine the presence of the HbA1c in the patient sample. In another example, the patient sample with the HbA1c is conjugated to a binder in a mixing chamber that is external to a cartridge, such as a microchip cartridge or other cartridge with an electrophoresis substrate. The conjugated HbAc1 can be prepared external to the system, such as in a mixing chamber that is lysed in a diluent for example or otherwise prepared for the immunoassay run. Once the bound HbA1c complex is conjugated and prepared for the immunoassay run, it is applied to the electrophoresis substrate 210.

The disclosed systems and methods have a voltage source that applies an electrical potential across two electrodes on opposing ends of the substrate on which the bound HbA1c complex is applied to cause the bound HbA1c complex to migrate across the substrate 212. The electrodes can be integrated into a cartridge on either side of and electrically coupled to the substrate on which the bound HbA1c complex is applied or can be electrodes within a reader into which the substrate with the applied bound HbA1c complex is inserted to run the immunoassay. The voltage source applies an electric potential across the electrodes, which causes the molecules or complexes with negative charge to migrate towards the positive electrode. In an example, as discussed above an electric potential of 250V is applied across the electrodes.

The disclosed systems and methods determine a migration velocity of the bound HbA1c complex as the bound HbA1c complex migrates across the substrate over time 214. The systems and methods determine the migration velocity by imaging or capturing video of the substrate before, during, throughout, and/or after the immunoassay run. The images and/or video taken over time are analyzed to identify a band of interest—a calibrator, the bound HbA1c complex, total Hb, HbA2, etc.—at a first time and location and a subsequent time and location or multiple subsequent times and locations. The movement of the band of interest over time is used to calculate the migration velocity for the band of interest. The migration velocity is determined for any one or multiple bands that migrate in the immunoassay. In some examples, the disclosed systems and methods determine a migration velocity for each band applied to the substrate.

As shown in FIG. 2B, the disclosed systems and methods detect a bound HbA1c complex band during, throughout, and/or after the HbA1c migrates across the substrate in the immunoassay run 216. The bound HbA1c complex band is visible or optically detectable on the substrate as it migrates. In some examples, the bound HbA1c complex band is visible to a human eye or the disclosed systems and methods can image and/or capture video of the substrate before, during, throughout, and/or after the immunoassay run. Sometimes an optical detection aid, such as a light of a specific wavelength like UV light is used to excite the HbA1c. If visible, with or without an optical detection aid, then a human user can observe the migration of the bound HbA1c complex on the substrate. In another example, the bound HbA1c complex is not visible or is optically enhanced to improve visibility of the bound HbA1c complex, such as imaging or capturing video of the substrate as it is exposed to UV light transmission. A human user or the disclosed systems and methods can detect the bound HbA1c complex any number of times throughout the immunoassay run. The disclosed systems and methods also can output data that includes the determination of presence of the HbA1c 220, as discussed above.

As described above, the disclosed systems and methods can determine presence of the HbA1c in the patient sample based on the bound HbA1c complex band and one or both of the migration pattern and a feature or characteristic of the migration velocity 218. A feature or characteristic of the migration velocity is an absolute or relative value of the velocity of a target compound band as it migrates across the substrate over time during an immunoassay run; whether the migration velocity increases, decrease, delays a start, or pauses during the immunoassay run; and the like. The migration velocity can be translated into a signal or graph representing the migration velocity of the target compound over time. The feature or characteristic of the migration velocity can also include a signal or graph characteristic, such as a slope, peak value, peak width, and the like.

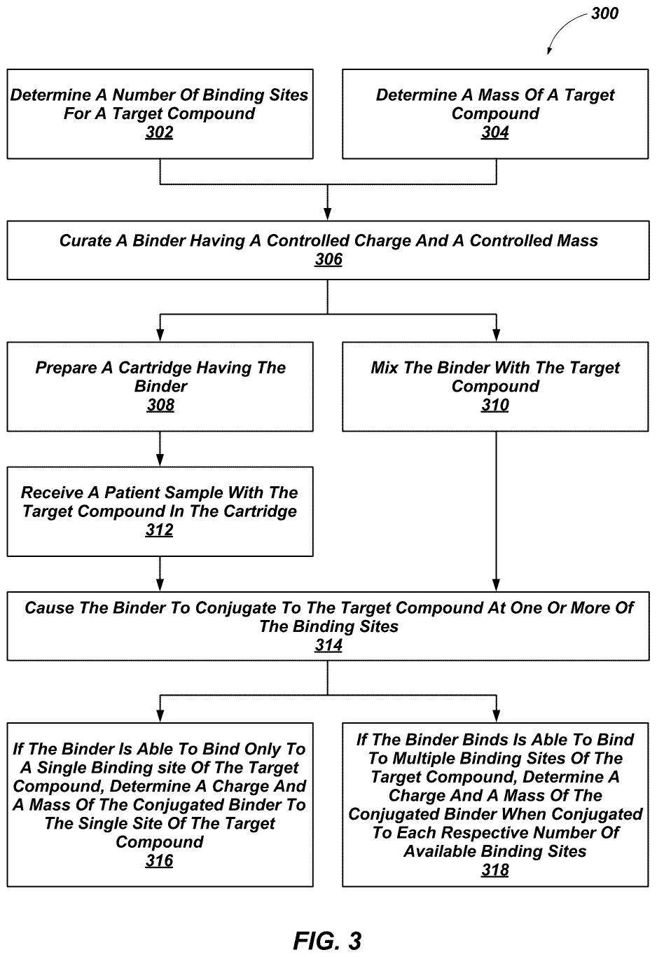

FIG. 3 shows an example method of curating a binder having a controlled charge and/or mass and conjugating the binder to a target compound. The target compound can be any target compound of interest. In the examples discussed above, the target compound is HbA1c although it can be any other protein, molecule, and/or biomarker that indicates the presence or absence of a disease state or metabolic condition in the patient sample. Each target compound has a specific number of binding sites to which a binder can conjugate and a mass. The disclosed systems and methods determine the number of binding sites for the target compound 302 and also determine a mass of the target compound 304. In some examples, the disclosed systems and methods determine the number of binding sites and the mass for multiple target compounds of interest. The numbers of binding sites and/or the mass of each target compound can be determined by empirical data, a library of past data, or newly acquired measurement data for the number of binding sites and/or the mass of the target compound.

The disclosed systems and methods curate a binder having a controlled charge and a controlled mass that is based on the target compound to which it is intended to conjugate. The curation process can occur either with a human use that curates the binder in a separate process than the data analysis. In some other examples, the same processing circuitry may include a module that identifies a known charge and mass of a target compound with a recommendation on features and/or characteristics of the binder. Such data can be output to a remote server, user interface module, or display to then begin the binder curation process. As discussed above, the curated binder can be prepared on a cartridge 308. In this example, the bound target compound complex is applied to or received in the cartridge, such as being applied to a well on a microchip cartridge to which a patient sample is applied 312. The binder can also be mixed with the target compound 310 prior to being applied to the electrophoresis substrate. The mixed target compound and binder is the bound target compound complex in which the disclosed systems and methods cause the binder to conjugate to the target compound 314. FIG. 3 also shows that the disclosed systems and methods determine a charge and a mass of the conjugated binder to a single binding site of the target compound 316 or to multiple binding sites of the target compound 318, depending on the number of available binding sites.

Turning now to examples of the disclosed systems and methods that detect infectious diseases such as COVID-19 and hepatitis C, the level of antigen available for detection of disease or detection of the pathogen is very low, which produces low sensitivity and specificity in standard lateral flow tests. For example, the required antigen level for hepatitis C is around 3 femtomoles (fmol) per liter. This concentration of target compound in the patient sample conjugates with the binder to form a bound binder-target complex having a total net charge and a mass. The binder is a molecule that attaches to the target compound to create the bound complex—oftentimes, the binder, rather than the target compound is the detectable component of the sample. For example, if the target compound is an antigen, such as a protein produced by or produced in response to a virus, the binder is an antibody conjugated to the antigen or an epitope of the virus protein in this example.

In these examples in which the volume of the target compounds is low (whether or not it is an infectious disease or a non-infectious disease, disease state, or metabolic condition) or in which visibility of the target compound(s) is low regardless of the volume, a label can be attached to either the binder or the target compound to produce a labeled bound complex—labeling of the binder or compound can occur before or after the binder and target compound are conjugated. This prepared patient sample includes a labeled bound binder-target complex. Patient samples without the target compound—for example, healthy patient samples or patients for whom the absence of the target compound or biomarker indicates a disease state—results in a prepared patient sample that includes unbound binder with its label, which can be either a substance visible in white light like a dye or a fluorescent label or “fluor.” No bound complex is present in the healthy patient sample, in some examples in which positive identification of the target compound indicates disease. In other examples, absence of the target compound indicates the disease state itself, such as in some hemoglobin disorders. The presence of the label on the bound complex allows for small concentrations of bound complex to be detected because the label is detected in the captured image rather than the bound complex itself. In some examples, the label is a substance that is visible in white light. That substance becomes attached to the labeled bound complex and is then visible on the captured image(s). The label increases the ability of the optical imager to detect presence of the label in small concentrations of bound binder-target complex. Even if the target compound band is not visible, the label may be visible and then becomes the molecule indicating presence of the target compound.

The label can also be a fluorescent label or “fluor” that responds to an excitation energy with a fluorescence emission. A fluor is a molecule that responds to excitation by an energy source such as light from a light-emitting diode (LED) or energy from a laser, with a responsive energy emission of its own. Many materials naturally have a certain fluorescence in response to an excitation energy, such as light at a particular wavelength. Some materials can be excited to fluoresce with a responsive energy emission of a particular behavior that is well defined enough to separate them from the background materials. The energy emission of the fluor is a different wavelength or range or wavelengths from the light or energy emitted from the excitation source. Often, the fluor has a time delay, in the nanosecond range in some examples, in which it then emits its fluorescence even after the energy source of excitation is turned off or removed. Certain compounds such as europium can persist for 600 microseconds or longer after excitation begins or is removed. The fluorescence emission from the fluor appears in the captured images. Without tight control over the charge state and the mass of the bound complex to which the label is attached, the images of a single type of labeled bound complex would migrate at multiple velocities, which causes a smear because of the different migration velocities corresponding to the different molecules or would produce results that could not be used to discriminate whether the target compound was present.

In an example, the fluor is attached to the binder. The binder conjugates to the target compound upon mixing with the patient sample. Unbound binder remains in the sample as well. Both the bound complex and the unbound binder are fluorescently labeled in this example. The bound complex is separated into its charge states. A selected charge state of the labeled compound is important to allow electrophoresis to occur without confusion between bands—there is clear band separation because molecules of like charge and mass migrate together—and the fluor allows for small concentrations of the target compound to be discovered and appear as a band in the images captured by the optical imager. Together, the fluors on the charge- and mass-controlled target compounds create clear bands of target compound in small concentrations using electrophoresis.

Further, photobleaching the substrate reduces the natural auto-luminescence of the substrate and further enhances the band detectability. Other components of the system can also be photobleached to reduce or eliminate their auto-luminescence. Photobleaching can occur during manufacturing or right before the test. Typically, the substrate or other system components are photobleached during manufacturing—in the example of the substrate—or prior to the test being run. Photobleaching reduces the auto-luminescence of the substrate, which means that the fluors on the bound complex appear brighter or to have higher relative intensity in the captured image(s). Photobleaching involves illuminating the substrate for a period of time with intense light, which can be a single wavelength or multiple wavelengths like high intensity ultraviolet (UV) light or broadband white light, to reduce the autofluorescence of the substrate. By reducing the auto-luminescence, the space between bands is darker on the substrate, which allows detection of a fainter band because its intensity is more detectable against the darker background. Reducing surrounding natural auto-luminescence of the substrate increases the differential of the band intensity of the bound complex to its substrate and to the intensity of other bands. Further, controlling the wavelength of the light from the energy source used to excite the fluors helps control the auto-luminescence of the substrate, for example. Many substrate materials, such as cellulose acetate and nitrocellulose have increased autofluorescence when illuminated with ultraviolet, blue, or green light and responsively emit blue or green light. Working with excitation light in the red wavelength range with emissions in the red or infra-red range minimizes much of this autofluorescence. Choosing a fluor with these characteristics reduces the impact of autofluorescence of the substrate and other components.

Conventional electrophoresis does not generally use fluorescence since staining and washing is generally used instead. While suitable for central laboratory work, conventional electrophoresis is slow, labor intensive, and does not allow imaging during migration, which results in the loss of important information about the compounds and biomarkers in the patient sample. Conventional lateral flow fluorescent immunoassays do not consider molecular charge because the flow or molecule separation occurs without application of an electric potential. The disclosed methods and systems that include a labeled bound complex overcome these challenges of the conventional methods to improve sensitivity and specificity and to lower the volume of detectable compound concentration required for the tests.

In typical electrophoresis, a fluorescent dye used prior to electrophoresis would cause a bleeding of results from molecules of varying charge states or what appears as a smear on an imaged substrate. This undesirable result previously prevented electrophoresis from being used with fluorescently or other labeled binders or compounds during the immunoassay run. In capillary electrophoresis, for example, the compound detector is positioned at a fixed location along the capillary tube and measures compound as it moves through the fluid or gel past the detector.

In this disclosure, the compound charge state of the bound complex is the total net charge of the bound binder-target complex,—including the binder charge and the compound charge—which is uniform throughout the entire prepared patient sample to give the bound complex of the patient sample a uniform or “controlled” total net charge. If the conjugation of the binder and the target compound is not controlled at a fixed ratio, the charge is not uniform but instead has various discrete values that cannot be discriminated using electrophoresis. The prepared patient sample is applied to an electrophoresis substrate, which is the medium on which the patient sample is placed for testing. In electrophoresis, the patient sample is applied to the test medium, which is often a paper or gel. Then, an electric potential is applied to the substrate across two electrodes positioned on either side of the applied patient sample. In standard POC electrophoresis and all paper or gel based electrophoresis techniques, the bound complex and all electrically charged components in the patient sample migrate across the substrate in response to the applied electric potential. All of the bound complex molecules generally migrate together, evenly or within a “band” toward the electrode of opposite charge because the total net charge and mass of the bound complex is generally uniform or controlled and thus drawn to the oppositely charged electrode with the same electric field force resulting in a uniform migration velocity.

The band is a collection of the component molecules that migrate with the same total net charge and the same mass. Small variations in mass or charge can be tolerated to still create a controlled band of molecules with the target compound. For purposes of this disclosure, “same” is intended to include small variations as well. This bound complex band is the collection of molecules of like charge and mass that have a common migration velocity. The band is the visual representation of the migrating molecules of like characteristics. The applied electric potential causes the molecules of like charge and mass to migrate or physically move a consistent distance at a consistent or uniform velocity. Because the small variations in charge and mass and other ambient environmental factors, some of the molecules with like charge and mass may have a migration velocity within a small range—the migration velocity of the molecules may slight vary. Here, reference to the “same” migration velocity also includes this small variation and still produces the disclosed controlled band that is optically detectable. The molecules of like charge and mass also migrate off the substrate or stop migrating if the electric potential is removed at about the same time and about the same distance—the migrating or stationary band of like characteristics is optically detectable on the substrate. In some examples, all of the migrating component bands are detectable by an optical imager. Some component bands may be difficult to optically detect or may not be optically detectable because their intensity is too low for detection. Labeling the bound complexes increases the intensity of the faint and undetectable bands so they become detectable by the optical imager. Other compounds produce a band with a higher opacity when labeled, which makes them easier to optically detect. This is especially true if the target compounds are all labeled with a fluorescent molecule.

The binder can be labeled or has intrinsic properties that allow it to be imaged. For example, the binder could be colorimetric, fluorescent, luminescent, or the like that is able to be optically detected when present in a band. These binders ensure consistency of charge and mass between each labeled binder to enable direct optical detection of the bands of concentrated bound complex and unbound binder during migration.

Examples of the disclosed disease diagnostic methods and systems are immunoassays that are performed on paper or gel electrophoresis substrates, such as cellulose acetate paper or agarose gel. In response to the applied electric potential, components of the patient sample migrate at respective migration velocities across the substrate toward the electrode of opposite charge based on the respective total net charge or charge state and corresponding masses of the respective components. This migration of components over an interval produces a migration pattern, which is a physical movement of components across the substrate in response to the applied electric potential. The optical imager captures image(s) during and at the end of the interval that reflects the final migration pattern of each of the components in the patient sample. The interval is the period of time in which components applied to the substrate migrate across the substrate. Specifically, the interval starts when the electric potential is applied and ends when it is removed. Without an electric potential, the components do not migrate in a clinically significant manner although there may be some nominal movement.

Alternatively or additionally, the optical imager can capture image(s) during the active assay—when the electric potential is actively applied to the substrate and the active migration occurs—to gain more detailed data on the migration of components throughout the interval. For example, dynamic image capture during the active assay produces data about how a charged molecule behaves throughout the interval rather than merely at the end, which can generate trends in molecular behavior in response to the applied electric potential over the interval and in migration velocity which may help determine or validate existing test results. Migration velocity is the velocity at which a component migrates or physically moves across the substrate in response to the applied electric potential and may be constant or variable in response to the applied electric potential. The migration pattern can include the physical location of the resulting bands of components that migrated across the substrate along with the thickness of the bands, and separation between bands. The migration velocities can be absolute or relative to a control or marker with fixed chemical concentration that co-migrates with the sample.

Further, the migration pattern also can include the dynamic movement of the components as they migrate across the substrate over the interval. For example, the migration velocity of a component can be tracked over the interval. The migration velocity of one component can be compared to the migration velocity of another component or a known standard or reference substance. The migration velocity for any migrating component can be variable or consistent throughout the interval. The change in migration velocity might also be considered along with trends in the migration velocity, thickness of the band, movement of the bands with respect to each other, behavior of the band when no target compound is present, and the like can also be tracked and trends can be extrapolated. Adding dynamic analysis of the migration pattern, including all aspects of component migration characteristics, helps distinguish molecules from each other based on multiple aspects of their molecular characteristic(s) or behavior(s).

Mass of the bound compound is another molecular characteristic that can affect its migration pattern and characteristics, such as migration velocity. Varying the mass of the binder controls the total net mass of the bound binder-target complex. Heavier bound complex moves slower than lighter bound complex in response to an applied electric potential. If a heavier binder is desired for its characteristics, such as its fluorescence response, it moves slower when all other aspects are kept constant. When the binder is conjugated to the target compound, the mass and charge of the bound complex determine migration velocity. The bound complex always has higher mass than the unbound binder. If the charge increases when the binder conjugates with the target compound, the bound complex moves faster. If the charge is neutral and does not increase when the binder conjugates with the target compound, but the mass of the binder increases the total mass of the bound complex, then the bound complex moves slower due to the increase in total mass of the bound complex from the mass of the compound without the binder. In other examples, the negative charge of the bound complex and mass of the bound complex both increase. In this embodiment, the migration velocity of the bound complex is determined based on whether the negative charge or mass of the binder has a greater impact when the binder is conjugated to the compound resulting in a higher negative charge and total mass of the compound.

For example, the binder can be chosen to have much lower charge than the target compound that is being detected. When conjugated, this can result in a larger charge differential between the unbound binder and the bound complex, which means the bound complex has a much higher charge than the binder alone. This causes an increase in the differential of migration velocity of the bound complex to the unbound binder. Since the bound complex has greater mass than the binder, it moves slower if the charge is identical or within a small difference to the binder charge. The combined charge is chosen to either slow down the bound complex or speed it up. The change in charge does not negate the increased mass or a new band does not separate. Therefore, the binder charge is carefully selected to create a separate band taking into account the change in mass that inherently occurs with the bound complex molecule.

The disclosed methods and systems use an optical imager to capture relative comparisons of component migrations, either in a single instance (e.g., the end of the migration interval) or throughout the active assay, to determine presence of a target compound in a patient sample. This disclosure discusses determining presence of a target compound in a patient sample in several examples although it can equally apply to determining multiple target compounds in the patient sample using the same or different respective binders. In an example method or system with multiple target compounds, multiple unique binders are tailored for each of the respective target compounds. In another example with multiple target compounds, the same labeled binder conjugates with the target compound to produce a bound complex for each of the target compounds—each of those bound complexes have a different mass and charge because the respective target compounds have different mass and charge.

The binder charge, mass, or combination of the charge and mass can be chosen to change the migration pattern to differentiate a target compound from other compounds in a patient sample, including other target compounds in the case of a “multiplexed” test that is determining whether the patient sample contains more than one target compound. Additionally, binder fluorescence can be selected so that the fluors either excite at different wavelengths, emit at different wavelengths, or both in order to multiplex tests for multiple target compounds.

Presence of the target compound is a detectable amount of the target compound in the patient sample. Some samples from healthy patients have no target compound and would therefore produce only a band indicating the presence of the unbound binder without an indicator that the binder conjugated to the target compound. Further, the optical imager can also optionally capture continuous imaging segment(s), in some examples, during the active assay. The active assay migration pattern is imaged throughout the interval to evaluate the characteristic of the migrating component bands across the substrate, not at a single specific location. This differs from other electrophoresis methods that rely on a single detection point to measure migration of a component, such as capillary electrophoresis through a fluid or gel matrix. It also differs in that the optical imager captures image(s) or continuous imaging segment(s) during the active assay in addition to capturing the migration image at the end of the assay when the interval is expired. The imaging can use one or multiple wavelengths of illumination or fluorescence. For fluorescence, the bound complex, binder, or the target compound may be labeled with a fluorescent reporter, such as the fluors described herein.

As discussed above, adding a label to the bound complex helps to detect the bound complex or the label indicating the bound complex is present in the captured images. In an example, the label is a dye added to the bound complex prepared patient sample. The dye is visible in white light. When the optical imager captures images either at the end of the electrophoresis run or actively during the run, the labeled bound complex is easier to detect on the images with the label than it would be without the label. This permits smaller concentrations of the bound complex to appear on the images as a band because the labeled bound complex appears before the unlabeled bound complex appears, assuming the unlabeled bound complex is visible at any concentration. However, because most unlabeled bound complexes are not visible at any concentration, a label is required to detect the migration of the bound complex band. In the examples in which the optical detector captures image(s) during the active electrophoresis run, the labeled bound complex can appear mid-way through the interval of the run to be compared to its later band migration pattern or other bands that appear over time to identify or validate it is the labeled bound binder-target complex.

In another example, a fluor is attached to the binder or the compound before it becomes a bound binder-target complex. The result is a fluorescently labeled bound binder-target complex, which responds to an excitation energy source, such as a light source. The excitation energy source directs light toward the substrate throughout or during portions of the active electrophoresis run to cause the fluors to respond with its fluorescence emission. The fluorescence emission appears on the captured image(s) generally at lower concentrations of bound complex than would occur with a non-fluorescent optical label. This happens because of this responsive fluorescence emission. Because the fluorescence emission is optically detectable, analyzing the image(s) for presence of the fluor during the active assay allows for the detection of the presence of smaller concentrations of the bound complex throughout the run. Of course, the image(s) of the substrate at the end of the interval can be used in addition to or alternatively to the images of the active assay, as needed.

Capturing image(s) during an active immunoassay allows for analysis of more aspects of a migration pattern, such as migration velocity of one or more components. Each migrating component in a patient sample has a respective velocity profile at which is migrates across the substrate in response to the applied electric potential. The migration velocities have a high correlation to the strength of the total net charge and the mass of the components—i.e., its attraction to the oppositely charged electrode. For example, in a system with migration towards a position field, components with negative charges farther away from neutral or “0” migrate at a higher velocity than components with charges closer to neutral or “0,” and the more positive a charge the slower they migrate, or they may even travel in reverse. For example, a bound complex with a high charge state has the fastest migration velocity, which helps to differentiate it from other slower migrating components—such as unbound binder—for a relative comparison. The ability to evaluate the migration velocity as part of the migration pattern analysis helps to further differentiate a bound target compound from an unbound binder or other patient sample components. Conventional electrophoresis only evaluates the image(s) after the immunoassay test run is complete, not during the active assay. Analyzing the migration pattern in the disclosed systems and methods during the active assay helps increase the sensitivity and specificity of its results.

Conventional immunoassay binders may vary in the number of binding sites to which they conjugate on the target compound. For example, simply mixing binder with compound would produce a bound complex having a variety of “charge states” and mass due to variation in the number of binders that conjugate to the number of molecules of the target compound. Most compounds have a specific number of binding sites to which binders may conjugate to create distinct groups of bound complex of like charge and mass. The binder can be selected to substantially increase or decrease the total net charge of the bound complex—the total net charge is directly related to the migration velocity of the component. One binder may add to the total net charge of the compound at a first level while another binder adds to the total net charge of the compound at a second level. The second level could be substantially great than the first level, which drives a faster migration velocity due to its stronger response to the applied electric potential.

This variable number of binders bound to the binding sites of the compound produces a variable total net charge of bound complex—each binder has a binder charge profile that changes the total net charge and mass of the bound complex depending on the number of binding sites to which the binder conjugates. The binder charge profile is the net charge of the binder on its own. For example, if a binder has mass 5 and charge +2 and can bind with up to three compounds—the target compound having mass 7 and charge −2—the resultant bound complexes are: (1) mass 12—charge 0, (2) mass 19—charge −2, and (3) mass 26—charge −4. The first bound complex does not migrate since it has a charge of 0. The second and third bound complex would move in the opposite direction as the binder since their charges are negative. There would be three bands in this case for the bound complex. Labels amplify this phenomenon. Many binders can bind to 1-4 labels. If the label, which has negligible mass, has a charge of +1 we now have 12 bands of bound complex for this example binder-target compound complex. These bands are often close together creating the appearance of a smear since the bands cannot be individually viewed.

By targeting only a bound complex with a specific total net charge and mass—compounds with binder conjugated to a fixed number of binding sites on the target compound and with a fixed number of labels—the bound complex can migrate across the substrate at a consistent migration velocity or within a desirable range of migration velocities. Without such control over the total net charge and mass of the bound binder-target complex, the bound complex of varying total net charge and mass migrates at different migration velocities across the substrate, which creates a smear on the substrate rather than clear bands, which, at a minimum reduces the sensitivity of the assay. Smears make obtaining diagnostic results difficult or impossible.

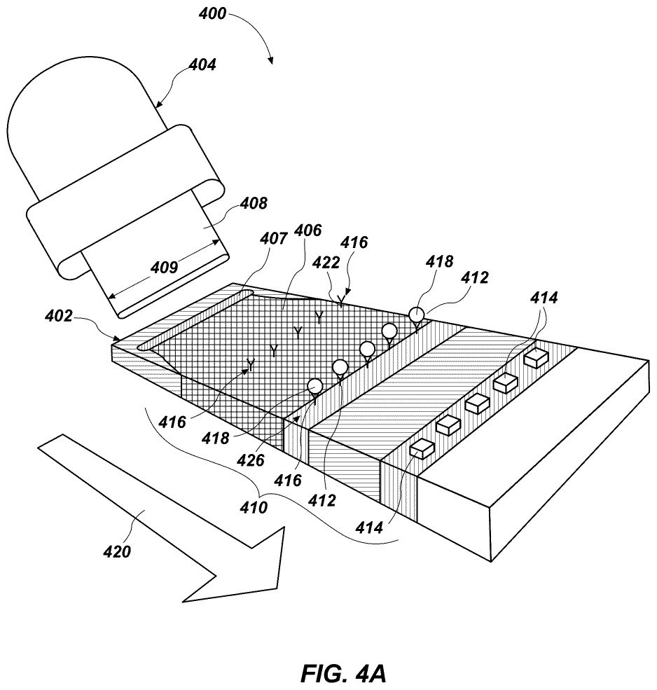

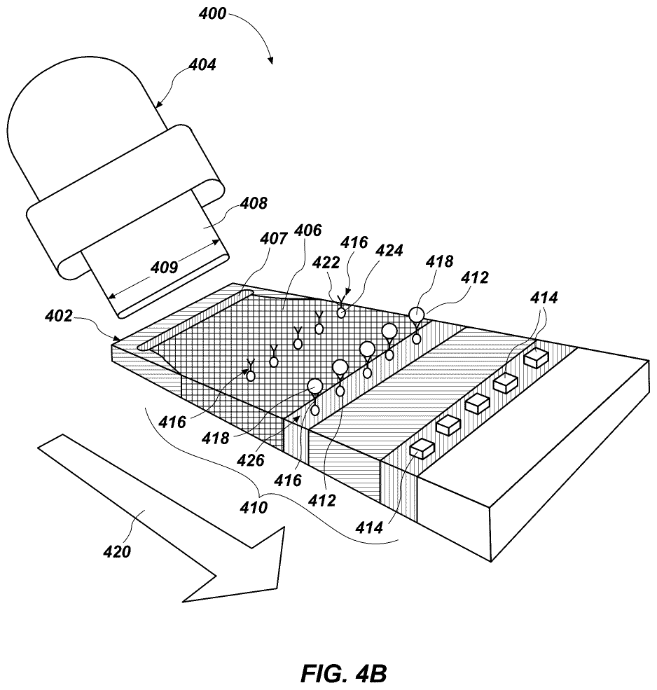

Further, after the patient sample has a bound binder-target complex, the bound complex can be separated into its respective charge states that correlate to the charge intensity or total net charge of the charge of the bound binder-target complex. For example, a fluor with a neutral charge is selected or a fluor with a specific charge state is selected. If a fluor with a neutral charge state is selected, then the labeled binder has the same charge state irrespective of the number of fluors attached to each binder molecule. In this example, the labeled binder would have a slightly heavier mass with the conjugation of the label(s) although that additional mass is negligible because the mass of a label is small compared to the overall mass of the binder molecule. Alternatively, the fluor is selected with a specific charge state, which is discussed in more detail below in reference to FIG. 4. More complex fluors with multiple charge states need to be filtered before they are incubated with the patient sample. The filtering of the fluorescently labeled binder results in a concentration of labeled binder of the same charge state and mass, which is then incubated with the patient sample. For example, fluors could have a charge of −3, −6, and −9—three distinct charge states—that are then fractionated before or after conjugating with the binder to a single charge state for the fluors or the fluorescently labeled binder, respectively. Even further, a binder can be chosen that has a single binding site on the target compound so that its charge state can only change to an expected bound charge state when the fluor conjugates to the binder—only one binding site is available to which the fluor can conjugate.

Some examples of the disclosed methods and system also include a fluorescent label or optical reporter that labels the binder that conjugates with the target compound. An optical reporter is optically detectable at one or more wavelengths but does not necessarily include fluorescence. A fluorescent label is a molecule or compound that “fluoresces” or glows at an emission wavelength(s) in response to excitation from an energy source, such as a laser or a light-emitting diode (LED) at a different wavelength than the emission wavelength. The fluorescent emission of the fluorescence label is in response to its excitation. The fluorescence emission of the fluorescence label occurs for a period of time, which can continue for a time after the excitation source is turned off. For example, europium is used as a fluorescent label or fluor in the disclosed methods and systems. Europium is typically excited at around 365 nm and has peak emissions around 610 nm. It continues its emission for at least 600 microseconds after the excitation source is turned off. Alternatively, other fluorescent labels can also be used, such as atto 665, Rho14, and alexafluor 488.

Alternatively, the target compound itself is labeled with the fluorescent label or optical reporter. Either way, the resulting bound compound in a prepared patient sample has a label. During or after the migration of the target compound and the other compounds in the patient sample, a fluorescence exciter—such as a laser or LED—is directed toward the substrate to cause the fluorescent label to “fluorescence” or emit its responsive transmission of light or to illuminate the fluor or optical reporter. For fluorescence, the labeled bound compound is detected by a fluorescence detector, which can be integrated with the fluorescence exciter in some examples. When florescence of an expected wavelength is detected, the target compound to which the fluorescent label is bound is known to be present.

Combining this optional fluorescence detection technique with the technique to evaluate the migration pattern of a target compound—possibly relative to other migrating components in the patient sample—is a powerful tool to create highly-sensitive, highly specific diagnostics using small concentrations of the target compound. As mentioned above, this concept extends to detecting multiple target compounds as well as detecting one target compound.

FIGS. 4A and 4B show an electrophoresis test 400 that includes a substrate 402—in this example the substrate 402 is a cellulose acetate paper strip—and a patient sample applicator 404—in this example the applicator 404 is a stamper. The stamper 404 contains or stores a prepared patient sample 406 that is applied to the substrate 402. The prepared patient sample 406 is applied to the substrate 402 along a compact application line 407. The compact application line 407 places all molecular components in the prepared patient sample within a small physical space to begin the migration process. If all of the charged molecules begin at this controlled application line 407, then their respective migration begins from that line and can progress together until the compounds of differing charge, mass, or both begin to separate during the migration process. If instead, the patient sample is applied to the substrate as a blot onto the substrate or deposited as a droplet onto the substrate, the molecules of like charge and mass migrate at the same migration velocity but begin from different starting positions. The different starting positions of molecules with like charge and like mass produce a thick band or do not appear as a band at all and transform into a smear on the substrate.

The patient sample chamber 408 of the stamper 404 has a width 409 that extends across the much of the width of the substrate 402 to produce the compact application line 407. The patient sample chamber width 409 can be just slight less than the width of the substrate 402 itself to create the application line 407 across most of the width of the substrate 402. Because the application line 407 extends across most of the width of the substrate 402, the band begins to form across the width of the substrate 402 when the molecules start to migrate in response to the applied electric potential. FIGS. 4A and 4B depict an ideal test in which molecules of like charge—the binder 416 (FIG. 4A), the binder 416 with a label 424 (FIG. 4B), the bound complex 426, and the marker compound 414—migrate perfectly aligned with each other. In a practical environment, a small migration variation can occur between molecules of like charge and like mass due to nominal variations in charge or mass between molecules and the exact placement within the application line 407. While the application line 407 is a practical attempt to apply the patient sample to the substrate in a way that allows all molecules of like charge and like mass to begin to migrate from the same physical location on the substrate, it is not possible for it to be exact but is instead has an inherent thickness, however narrow. The inherent thickness of the migration of molecules of like charge and like mass correlates to the thickness of the resulting band after the electric potential is removed.

In this example, a patient sample chamber 408 of the stamper 404 releases the prepared patient sample 406 onto the cellulose acetate paper strip 402 at the application line 407. This cellulose acetate paper strip 402 shows a migration pattern 410 of a bound complex 412 and a marker compound 414 after an electric potential has been applied to the substrate 402 for an interval. Binder 416 is shown conjugated to the target compound 418 (the binder not labeled in FIG. 4A and is labeled in FIG. 4B) and separating into a bound complex band along the direction of migration 420. The bound complex has binder 422 with a fluorescent label 424 and a consistent or uniform compound charge state. For example, the binder 422 has a pH 8.3 and a charge of approximately −3 and the target compound has a neutral charge. This binder charge profile of −3 could conjugate to the target compound 418 in one to four binding sites—which creates four charge-mass profiles with different charge and mass—that would vary the total net charge of the bound complex 412 between −3 and −12 and correspondingly vary the mass of the bound complex 412 in the same manner. The binder 416 is designed and refined such that only a single (or a fixed) number of conjugations can occur. In an example, the fluor is selected to have a neutral charge that does not alter the charge state of the binder when the fluor conjugates to the binder. Further, because fluors are typically very low in mass, especially compared to binder molecules, adding neutral fluors to a binder does not substantially change the mass of the fluorescently labeled binder compared to the mass of the unlabeled binder. Alternatively, the disclosed methods and systems have a fluor with a specific charge state, which is able to be filtered or that can be fractionated to a single charge state before or after conjugating to the binder, such as by using anion exchange chromatography. Alternatively, a binder is chosen that only has a single binding site on the target compound so that its charge state can only change to an expected bound charge state when the fluor conjugates to the binder—only one binding site is available to which the fluor can conjugate.

During preparation of the patient sample, for example, the binder 416 conjugates with the target compound 418 in an incubation step that can be a short period of time, such as several minutes, to allow the sample to sit at room temperature to create the bound complex 412 that having a uniform, single charge and mass state. The uniform charge-mass state for the bound complex 412 moves across the substrate 402 with a migration velocity to form the bound complex band 426.

This example prepared patient sample 406 also includes marker compound 414, which is a controlled concentration of a molecule in a different charge-mass state than the charge-mass state of the bound complex 412 found in the prepared patient sample 406. It might also have different optical properties, such as different color(s). The marker compound is typically a different chemical than the target compound or the binder and has a corresponding marker charge and mass. The differential in the charge and mass of marker compound to the charge and mass of the bound complex produces images with intensities that can be compared based on the known properties of the marker compound. The marker produces a migration pattern distinct from the migration of the bound complex 412 or the unbound binder 416. The marker compound 414 in this example has a higher charge state—or more negative charge than the bound complex 412—that causes it to migrate faster along the substrate 402 than the bound complex 412. The maker compound 414 migrates further along the substrate 402 than the bound complex 412 because it is more negatively charged than the bound complex 412 so it is drawn more rapidly and thus closer to the positive electrode (not shown) by the applied electric potential.

The migrated marker compound 414 produces a marker compound band 428 that is a guide against which the other bands are compared. The marker compound 414 has an expected migration pattern because of its unique mass and charge compared to the target compound 418, the binder 416, or the bound complex 412. In response to the applied electric potential and because its charge-mass state is fixed and differs from the other molecules in the sample, characteristics of the bands produced by other molecules in the sample can be compared to it as a point of reference. The marker compound band 428 appears when the bound complex 412 is present and when it is not present in the prepared patient sample 406, which makes it a “marker” or guide to which relative comparisons of other bands are made, such as the bound complex band 426. Any other band that appears can be measured against the marker compound band 428 and the known characteristics of its migration pattern and migration velocity. The marker compound band provides a normalized reference and can also act as a control to ensure the electrophoresis process is working correctly with the velocity and intensity being within expected parameters.

In a patient sample with no target compound 418, the migration pattern 410 would include the marker compound band 428 alone without a bound complex band 426. The unbound binder 416 that is mixed with the patient sample has its own charge-mass profile, so it migrates when the electric potential is applied to the substrate 402 to create an unbound binder band (not shown in FIG. 4). The unbound binder band has distinct characteristics from the bound complex band in migration pattern—its migration velocity, distance, and band thickness, for example—and a distinct charge-mass profile. The unbound binder migration pattern and migration velocity differs from both the bound complex migration pattern and migration velocity and the marker compound migration pattern and migration velocity. Because the unbound binder has a known charge and migration profile, it is another marker or control band that can be used as a relative comparison to any bound complex band that appears. The binder, labels, fluorescent labels, pH, and other molecular characteristics of each component are chosen to ensure each of these elements creates a unique band distinct from the others.

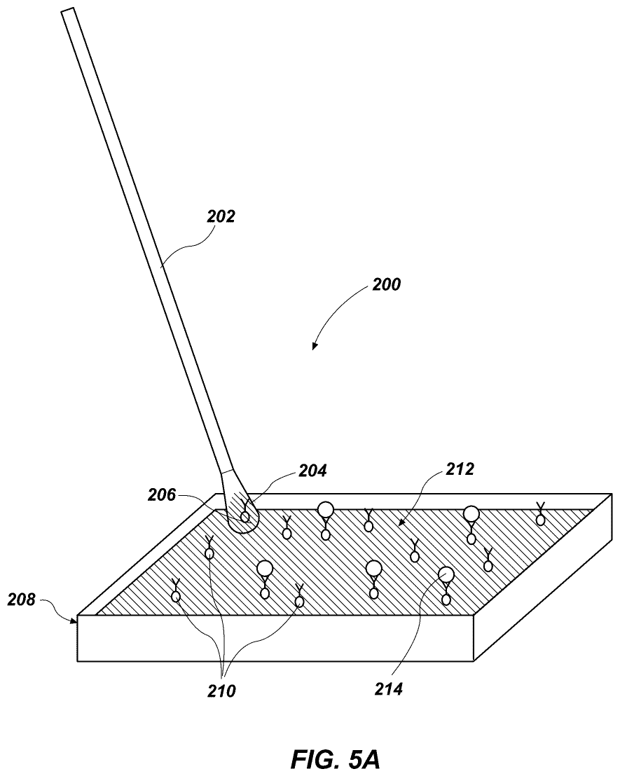

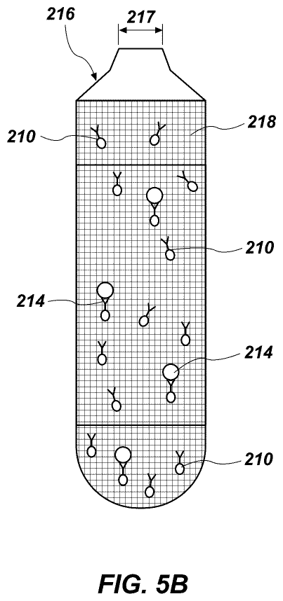

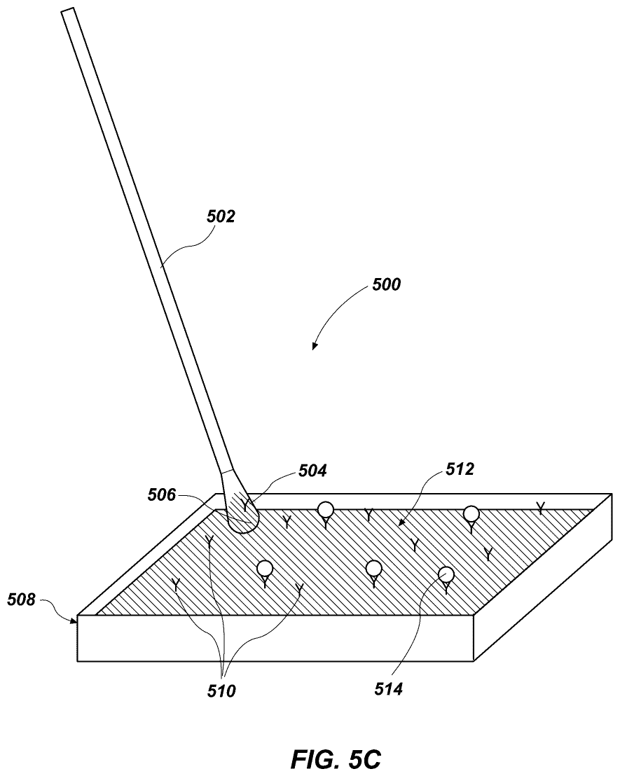

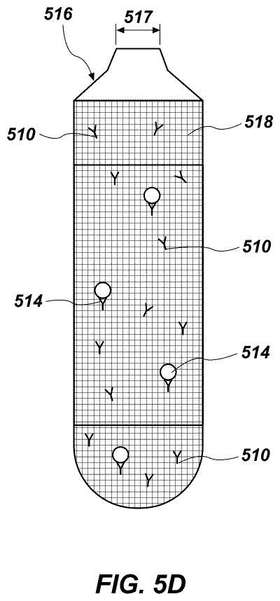

Turning now to FIGS. 5A and 5B, a patient sample preparation 500 has a patient sample collector 502 with a patient sample 504. FIG. 5A shows a patient sample 504 with the target compound 506 on the sample collector 502. The sample collector 502 in this example is a swab stick, but it could be any suitable sample collector and differs based on the type of patient sample being collected for the test. The patient sample 504 is being shown here to be placed in a preparation chamber 508 that has labeled binder 510. In this example, the label could be a dye or a fluorescent label, such as a fluor that emits after being excited at a light emission at an excitation wavelength. Some of the labeled binder 510 conjugates to the target compound 506 while some of the binder remains as unbound binder 510 in the prepared patient sample 512. FIG. 5A shows a simple preparation chamber 508, and any suitable preparation chamber 508 could be used, including those that are integrated into a reader or other diagnostic system equipment or into a cartridge that prepares the sample for insertion into a reader or other portion of a diagnostic system. FIG. 5A shows a prepared patient sample 512 with unbound binder 510 and labeled bound complex 514 that produces a positive result for presence of the disease or biomarker.

FIG. 5B shows a cartridge 516 having a width 517 of its applicator tip that contains the prepared patient sample 512 as it incubates with unbound binder 510 and labeled bound complex 514. The width 517 of the applicator tip applies a tight application line of the patient sample on the substrate. In some examples, the unbound binder 510 is preloaded into the cartridge 516 in a preparation chamber 518 of the cartridge 516, and the patient sample 504 is added to the preparation chamber 518. The labeled unbound binder 510 conjugates with the target compound 504 in the preparation chamber 518 of the cartridge 516 in this example. The cartridge 516 can be inserted into a reader that includes the electrophoresis equipment, such as the paper onto which the prepared sample is applied, the electrodes for applying the electric potential, the optical imager, the light source, the processing circuitry to run the electrophoresis test, and the like. In other examples, the cartridge 516 itself has an integrated substrate on which the prepared patient sample 512 from the cartridge preparation chamber 518 is applied. The cartridge 516 can have an integrated patient sample collector (not shown) or have an opening into which the patient sample 504 is added to the preparation chamber 518.

FIGS. 5C and 5D show a patient sample preparation 500 with a patient sample collector 502 with a patient sample 504. FIG. 5A shows a patient sample 504 with the target compound 506 on the sample collector 502. The patient sample 504 is being shown here to be placed in a preparation chamber 508 that has binder 510 without a label. Like in the example discuss above, in FIGS. 5A and 5B, some of the unlabeled binder 510 conjugates to the target compound 506 while some of the unlabeled binder remains as unbound binder 510 in the prepared patient sample 512. FIG. 5D shows a cartridge 516 having a width 517, like the cartridge shown in FIG. 5B, of its applicator tip that contains the prepared patient sample 512 as it incubates with unbound binder 510 and unlabeled bound complex 514.

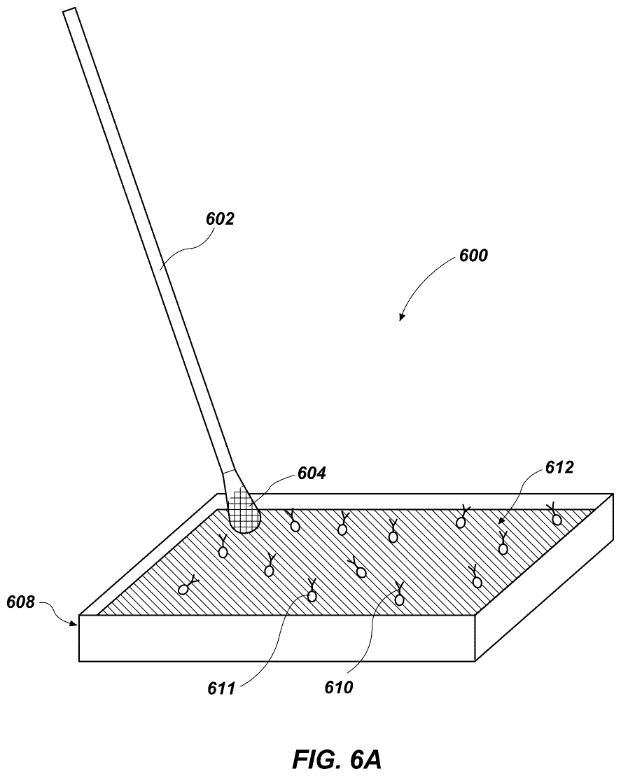

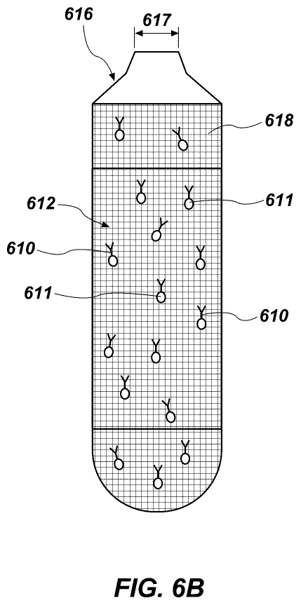

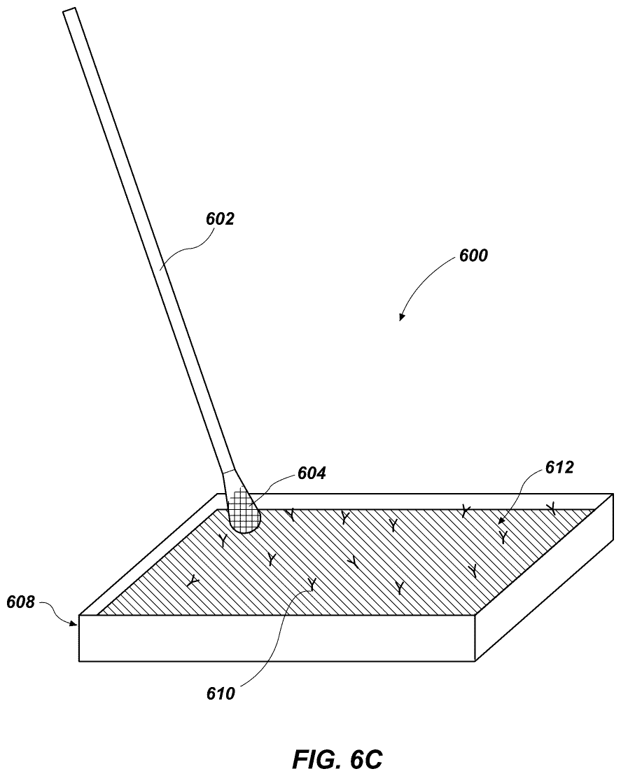

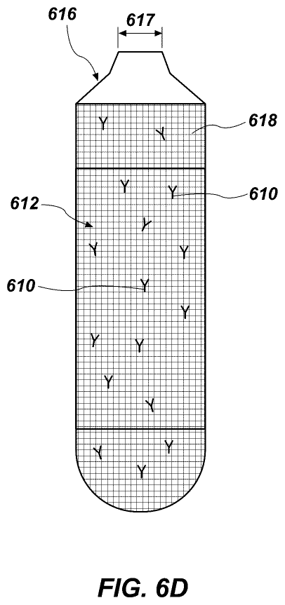

FIGS. 6A and 6B show a patient sample preparation 600 with a patient sample collector 602 with a patient sample 604 for testing, but unlike FIGS. 5A-5D, this patient sample 604 has no target compound—it is, for example, a healthy patient sample and produces a negative result for presence of the disease or biomarker for those diseases or pre-disease conditions in which presence of the target compound indicates disease or an be a positive sample if the disease state or pre-disease condition is detected by an absence of a target compound, such as in some hemoglobin disorders. FIG. 6A shows the patient sample collector 602 with the patient sample 604 that has no target compound, and the preparation chamber 608 that includes unbound binder 610 with a label 611. FIG. 6B shows the prepared patient sample 612 of FIG. 6A in a cartridge 616. This prepared patient sample 612 only includes unbound binder 610 with a label suspended in the prepared patient sample fluid or tissue. The results of the applied electric potential only produce an unbound binder band imaging in this example. FIGS. 6C and 6D shows the prepared patient sample 612 that only include unlabeled binder 610 (no label 611).

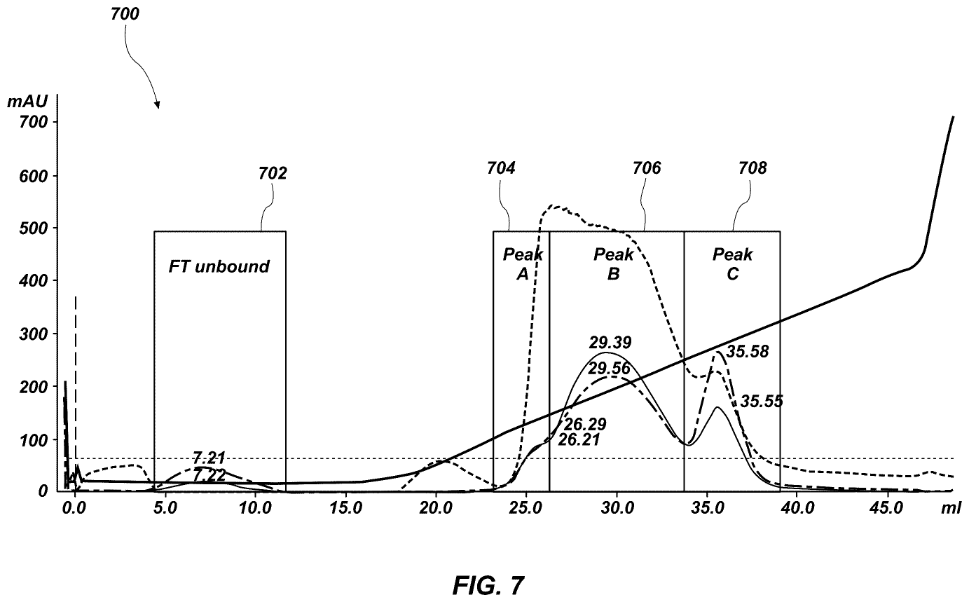

FIG. 7 shows a graph of anion exchange chromatography for a patient sample with unbound binder 702 and fluorescently labeled binder having three distinct charge states 704, 706, 708. As discussed above, a fluor or other label with a neutral charge can be selected, in some examples, or a fluor or other label with a specific charge state is selected, as in the example labeled binder molecule shown in FIG. 7. In the example graph shown in FIG. 7, a complex labeled binder is chosen that has three charge states 704, 706, and 708. Each of these charge states 704, 706, and 708 can be fractionated to the desired charge that needs to conjugate with the binder to produce the desired total net charge of the bound complex during incubation of the patient sample. Targeting a specific fluor with a known charge and then filtering the fluorescently labeled binder controls the charge and mass of the labeled binder. As shown in FIG. 7, binder molecules can have different charge states—in this example there are three charge states 704, 706, and 708—that correlate to the number of fluors bound to the binder. The three charge states 704, 706, and 708 of the labeled binder has a different charge state than unbound binder 702 alone.

The labeled binder would have a slightly heavier mass with the conjugation of the label(s) although that additional mass is negligible because the mass of a label is small comparative to the overall mass of the binder molecule. Alternatively, the fluor is selected with a specific charge state, which is discussed in more detail below in reference to FIG. 7. More complex fluors with multiple charge states need to be filtered before they are incubated with the patient sample. The filtering of the fluorescently labeled binder results in a concentration of labeled binder with a single charge state and mass, which is then incubated with the patient sample. For example, fluors could have a charge of −3, −6, and −9—three distinct charge states—that are then fractionated before or after conjugating with the binder to a single charge state for the fluors or the fluorescently labeled binder, respectively. Even further, a binder can be chosen that only has a single binding site on the target compound so that its charge state can only change to an expected bound charge state when the fluor conjugates to the binder—only one binding site is available to which the fluor can conjugate.

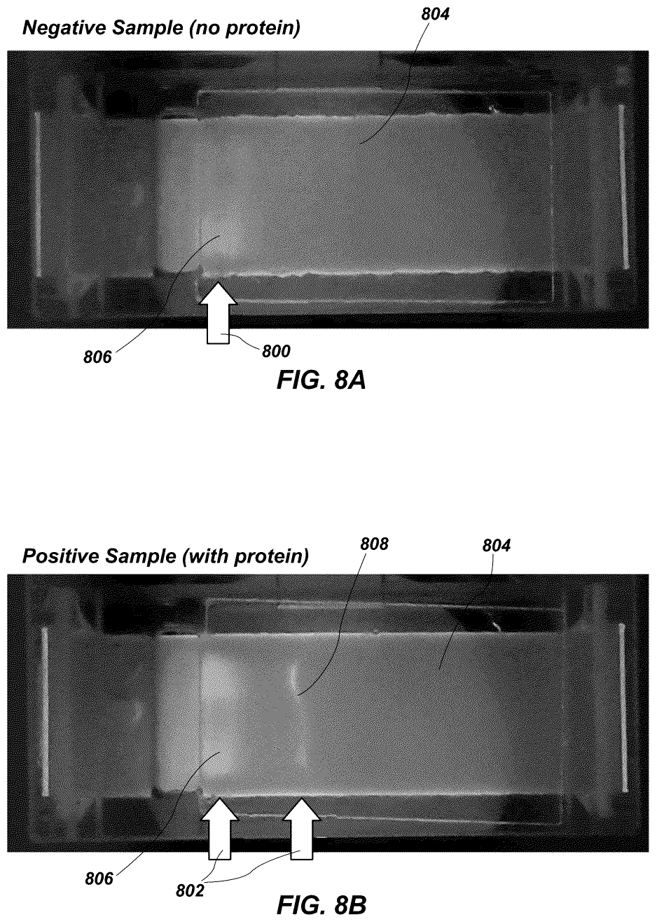

FIGS. 8A and 8B show migration patterns 800, 802 on a substrate for a negative sample—having no presence of the target compound—and a positive sample—having presence of the target compound—respectively. FIG. 8A shows a sample applied to an electrophoresis paper with an unbound binder band 806 that appears near the site on the substrate where the prepared patient sample is applied. No marker compound is included in this example. The patient sample has no target compound. The only band that appears is the unbound binder band 806. Because its migration pattern is known and no bound complex band appeared, this patient sample can be deemed to have had a successful run (the unbound binder band appeared with an expected migration pattern) and is negative for the target compound (no bound complex band appeared in the successful run). FIG. 8B shows the same unbound binder band 806 in approximately the same location on the substrate—in its expected migration pattern based on its known and fixed mass and charge state. FIG. 8B also includes a target compound band 808 that has a migration pattern that differs from the unbound binder band 806. The target compound band 808 migrated further and is thinner or is a “tighter” band than the unbound binder band 806, for example. The comparison of these and other characteristics of the migration pattern of the bound complex band 808 to the migration pattern of the unbound binder 806 indicates presence of the target compound in the patient sample, which is a positive or infected sample. Additionally, the intensity of the target compound band 808 could be analyzed to determine quantification values, such as its concentration, which is discussed in more detail below.

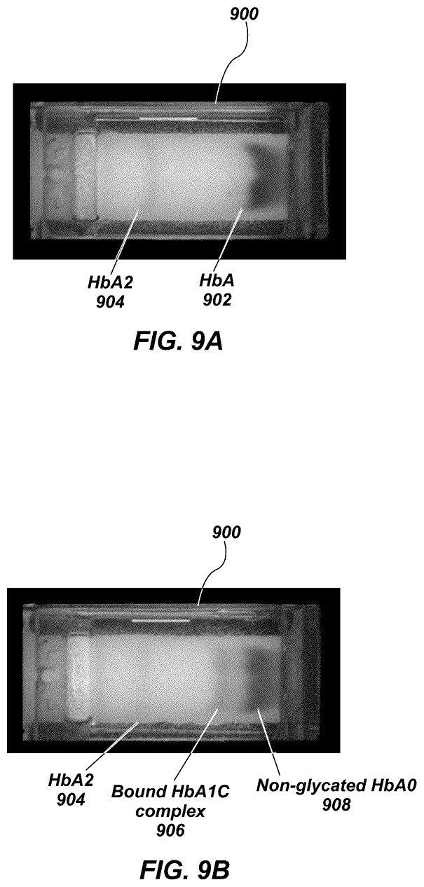

FIGS. 9A and 9B show migration patterns 900 on a substrate for a sample with the target compound of HbA1c. In FIG. 9A, the first step of separating total Hb from the patient sample occurs. Here, HbA2 904 and HbA 902 are shown for a patient sample with no HbA1c antibody or binder present. In the example shown in FIG. 9A, the HbA1c (not separately shown) co-migrates with HbA 902 so it is not separately detectable. This example shown in FIG. 9A is a typical hemoglobin electrophoresis for a healthy individual that does not separate and is not capable of separating out the HbA1c or other Hb sub-types. FIG. 9B shows the second step of separating components of the total Hb in which the antibody or binder for HbA1c is present. In this example, the bound HbA1c complex 906 separates from the total Hb, including the HbA2 902 and the non-glycated HbA0 908 as it migrates across the substrate. The bound HbA1c complex 906 is able to separate from the HbA2 902 and the non-glycated HbA0 908 in the disclosed systems and methods because the antibody changes the total mass of the bound HbA1c complex (HbA1c conjugated to the antibody/binder) and changes the total net charge of the bounder HbA1c complex to cause the HbA1c complex to move slower than the HbA. The slower moving bound HbA1c complex results in 3 bands—HbA, HbA1c (conjugated with an antibody), and HbA2. In this example, the net impact of the increased mass and charge of the antibody bound to the HbA1c is to slow down the migration of the HbA1c -antibody complex (bound HbA1c complex) relative to the migration of the HbA. HbA1c co-migrates with HbA if the HbA1c in its unbound state. It is the conjugation of the HbA1c with the binder having a controlled charge and controlled mass.

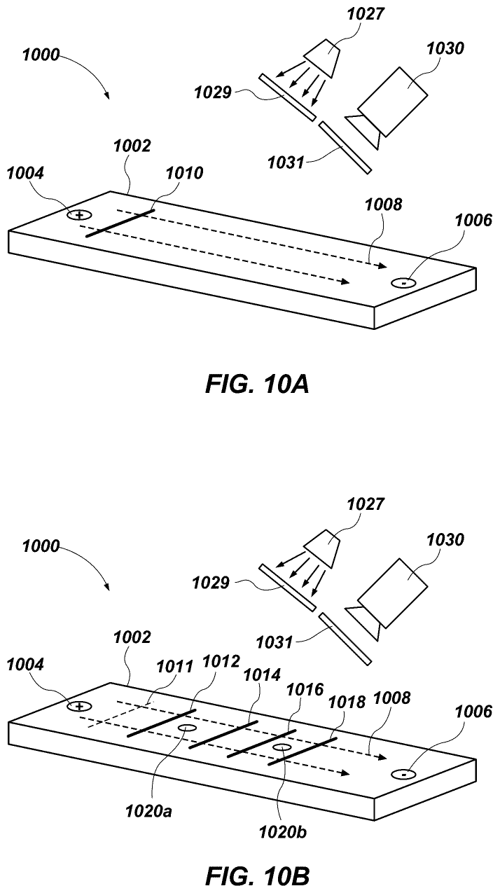

FIGS. 10A and 10B illustrate an example electrophoresis system 1000 that includes an electrophoresis substrate 1002, electrodes 1004, 1006, patient sample 1010 and an optical imager 1030. Results of the electrophoresis analysis are optically captured by the optical imager. A light source 1027 can be used to assist the capture of the results, with the light source 1027 emitting light that is then reflected or transmitted through the electrophoresis paper 1002 to help with imaging or visualizing the electrophoresis results on the paper. The wavelength characteristics of the light can be controlled by excitation filter 1029 and the wavelengths observed by optical imager by emission filter 1031. These filters are particularly important when using fluorescent labels to separate excitation light from fluorescent emissions.

FIG. 10A illustrates the initial set-up of the electrophoresis process. The electrophoresis paper 1002 can have a buffer solution deposited on it to assist with establishing the electrical conductivity between the two electrodes 1004 and 1006. A prepared patient sample 1010 is placed on the electrophoresis paper 1002 in a controlled manner in this example. In the example shown in FIG. 10A, the patient sample 1010 is shown deposited on the paper 1002 as a line. The precision or control with which the prepared patient sample is deposited onto the electrophoresis paper 1002 correlates with “tighter” bands in the migration pattern. If the sample begins from a relatively narrow starting location, its migration is more uniform than if it begins migrating from a smeared or spread out location. Tighter bands help in band comparison and analysis because the images are compared by pixels, band intensity, location, and width, which are all improved with the control over the migration of the compounds after controlled application of the prepared patient sample on the substrate. Additionally, the patient sample 1010 can include added compounds, such as one or more marker compounds, as discussed above.

For example, the one or more markers can have known migration rates or distances for a given applied voltage or voltage application time. Alternatively, these marker compounds can normalize the results of the electrophoresis process by having migration rates relative to the sample. Relying on relative migration rates, rather than absolute values, reduces the calibration needed between tests. Sample-to-sample variability makes absolute value determinations difficult because prepared samples, environmental conditions, and other test characteristics may vary or not be precisely the same between tests. The relative comparison serves to normalize this test-to-test calibration issue.

FIG. 10B illustrates the completed electrophoresis run of the patient sample shown in FIG. 10A. After applying an electric potential, via electrodes 1004 and 1006, for an interval, the patient sample 1010 has separated into the various bands, 1012, 1014, 1016 and 1018, which have moved from an initial patient sample location 1011. Additionally, added markers 1020a and 1020b have separated from the initial patient sample 1010 and have moved along the length of the electrophoresis substrate 1002. The intensity, location, thickness or other band characteristics or of the migration pattern of the various bands 1012, 1014, 1016 and 1018 can be used to identify the components, and their relative proportions, of the initial patient sample 1010. The optical imager 1030 captures image(s) of this migration pattern on the electrophoresis paper 1002 during or after the electrophoresis run. Those captured image(s) are processed to identify the compounds represented by the various bands 1012, 1014, 1016 and 1018 and their relative proportions.

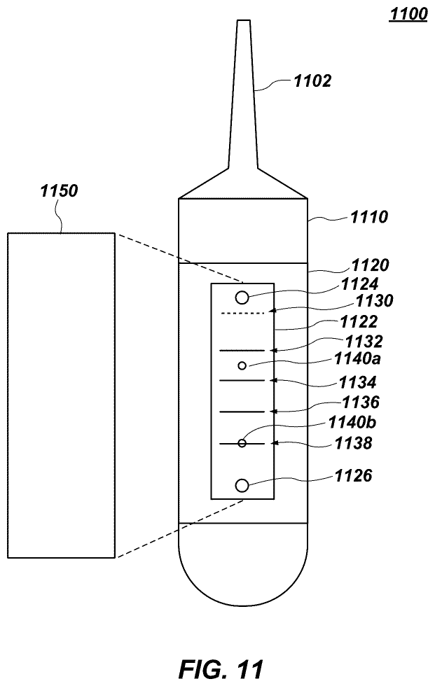

FIG. 11 illustrates an example cartridge 1100 and various patient sample analysis systems in relation to the cartridge 1100. The patient sample analysis systems can be included on a reader into which the cartridge 1100 is inserted or received, the cartridge 1100 can be inserted or received in a specific alignment or orientation in relation to the patient analysis systems of the reader. Additionally, one or more portions of or a complete patient sample analysis system can be included with the cartridge 100.

As shown in FIG. 11, the patient sample has separated into four distinct bands 1132, 1134, 1136 and 1138 in its migration pattern, which is shown through a window of the cartridge. Each band corresponds to a compound in the prepared patient sample. As discussed throughout this disclosure, the compounds can be identified based on their migration across the electrophoresis paper 1122. Additionally, markers 1140a and 1140b were included in the prepared patient sample to assist with the band analysis, as discussed above. In the example shown, marker 1140a is positioned between bands 1132 and 1134. The positioning of marker 1140a between the two bands 1132, 1134 can helps differentiate each of the bands 1132, 1134 during analysis of the band data to give a relative point of reference—marker 1140a—to clearly differentiate between band 1132 and 1134.

The other marker 1140b in FIG. 11 is shown positioned with band 1138. Marker 1140b was selected to have a different total net charge and mass as the target compound associated with band 1138. Band 1138 migrates to the same location as marker band 1140b, which indicates presence of the target compound because the total net charge and total net mass of the marker is known. The marker needs different optical characteristics to separate it from the co-migrating band. Any compound with a migration pattern that matches it, includes the target compound. The marker total net charge and total net mass are selected to intentionally match that of the bound target compound if it exists in the patient sample. For example, it could emit a different wavelength under fluorescence than the bound complex to allow both molecules to co-migrate but remain individually detectable.

Imaging of the electrophoresis substrate 1122 and the bands and markers thereon can be performed using a set or varying spectrum of light or optical imaging devices and techniques, to capture a variety of information for use in analysis of the bands. In various lighting conditions and spectrums, different aspects of the bands can be more easily ascertained, such as band position and intensity. Additionally, the markers can be selected to fluoresce in response to excitation lighting, which makes it easier to determine a position of a marker relative to a band on the electrophoresis substrate 1122. Multiple different fluorescing labels could be used so they can be optically distinguished. Alternatively, imaging of the electrophoresis paper 1122 and the migration pattern can be performed using any suitable optical imaging device, such as a digital camera. The cartridge 1100 can be imaged using the digital camera, such as by a cell phone camera, and the captured image can be transferred to a device or system for analysis or evaluation. Such functionality can also be a secondary analysis, or verification, method to support the optical imager of a reader into which the cartridge is fitted for the assay.

Because the bound complex is labeled, in this example with a fluorescent label or “fluor,” the system in FIGS. 11A and 11B show a light source 1127 with a filter 1129. The light source 1127 is the excitation energy source that emits light towards the sample to excite the fluors attached to the bound complex. In the example shown in FIGS. 11A and 11B, the light source 1127 is a white light source with a filter 1129 that permits only an excitation wavelength of light from the white light to be transmitted towards the substrate to excite the fluors conjugated to the bound complex. The filter of the light source can vary the wavelength, as needed, which allows it to transmit a range of wavelengths at the substrate that can excite a variety of fluors. In these examples, the optical imager 1130 may also have a filter 1131 that only permits light of the wavelength emitted by the excited fluors after excitation to the optical imager. In other examples (not shown), the light source 1127 emits light of an excitation wavelength for the fluors and does not need a filter. In this alternative example, the optical imager can still include a filter to target the wavelength of light emitted by the excited fluors.

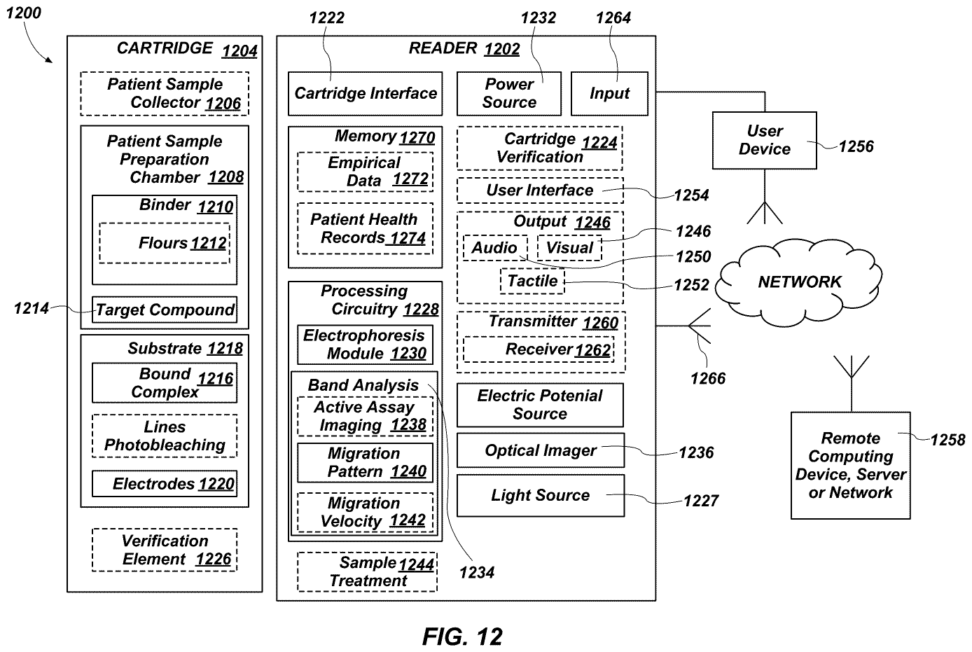

FIG. 12 illustrates an example system 1200 with a reader 1202 and a patient sample cartridge 1204. The reader 1202 can include all or a portion of the required systems or elements required to perform analysis of a patient sample. The cartridge 1204 includes a patient sample collector 1206 that could either directly collect the patient's sample or could receive a collected patient sample to facilitate the patient sample being added to the patient sample preparation chamber. In the example shown in FIG. 12, binder 1210 with conjugated fluors 1212 is included in the patient sample preparation chamber 1208. Alternatively, the binder 1210 can be unlabeled and do not include labels and/or fluors entirely. It is mixed with target compound 1214 so it conjugates with the patient sample to create a bound complex with fluors 1216, as discussed above. The fluorescent bound complex 1216 is applied to the substrate 1218, which is also included in the cartridge 1204 in this example. This example cartridge 1204 also has electrodes 1220 coupled to or placed on the substrate 1218 for applying the electric potential after the cartridge 1204 is inserted into a cartridge interface 1222 of the reader 1202.

The reader 1202 includes a housing that surrounds and encloses some portion or all of the reader components. Any one or more components of the reader can be external to the reader housing, as needed or desired. As previously discussed, the housing of the reader 1202 is constructed of suitable materials which may involve a suitably robust construction such that the reader is rugged and portable. Alternatively, the reader 1202 can be designed for use in a permanent or semi-permanent location, such as in a clinic or laboratory. Example materials that can be included in the housing include plastics, metals, and composites.

The housing can be constructed of multiple or a singular material and can include geometry or structural features that enhance the usability of the reader 1202. Such features can include a smooth outer surface that is easily cleaned, grips or handles for carrying the reader 1202, shock protection or increased structural strength in locations to prevent damage to the internal components of the reader 1202, insulation or heat dissipation structure(s) assist with maintaining a desired or a stable temperature, or range, within the housing, a membrane or construction to prevent the intrusion of moisture or dust into the interior of the reader 1202, connections, ports or interfaces for connecting the reader 1202 to an external element or device using a physical or wireless connection, instructions regarding the use of the reader 1202, identification markings such as a serial number or additional necessary or desirable features that can facilitate the safe, effective, efficient or proper use of the reader 1202. The housing can feature access points, such as removable or openable panels, to allow access to the interior of the reader 1202 for maintenance or repair of the internal components, elements or systems of the reader 1202. Additionally, the housing of the reader 1202 can be removable or separable from the other components, elements, or systems of the reader 1202, allowing the replacement of the housing, easing the cleaning of the housing, providing access to the components, elements or systems of the reader 1202 or other abilities that require or are made easier by the removal of the housing of the reader 1202.

The housing of the reader 1202 includes a cartridge interface 1222 that interacts with or engages the patient sample cartridge 1204 for analysis of a patient sample, such as the patient sample cartridge 1204 shown in FIG. 11. The cartridge interface 1222 can be a slot that is shaped to receive the cartridge 1204. The user inserts the cartridge 1204 into the slot in preparation for analysis of the patient sample. The slot can include internal geometry that aligns or orients the inserted cartridge 1204 in a proper alignment or orientation for the components, elements or systems of the reader 1202 to perform the requisite or desired analysis of the patient sample contained within the cartridge 1204. For example, the cartridge interface 1222 can accept a variety of cartridges having different cross-sections, such as square, rectangular, and circular cross-sections. The unique shape of each cartridge—the unique cross-section—can interact with the geometry of the cartridge interface 1222 to properly align the cartridge 804 within the reader 1202 for analysis.

The reader 1202 can also include a cartridge verification system 1224. The cartridge verification system 1224 can be integrated with or separate from the cartridge interface 1222 or can be included internal to or external from the reader 1202. The cartridge verification system 1224, in this example shown in FIG. 12, is integrated within the reader 1202 and detects a verification element 1226 associated with the cartridge 1204 when it engages with the cartridge interface 1222, such as a QR code, bar code, or other unique identifier. The cartridge verification system 1224 can verify the legitimacy of a cartridge 1204 to assist with efficient and effective analysis of a patient sample. Additionally, verification of the cartridge 1204 can include determining that the cartridge 1204 has not been previously used. The reuse of cartridges can be allowed or not, based on testing or manufacturer requirements, and the verification system 1224 can be used to enforce the desired or required limitation on reuse of a cartridge 1204. Once the cartridge 1204 is verified, further analysis of a patient sample contained within the cartridge 1204 can be allowed to proceed. The verification of the cartridge 1204 can be the threshold analysis of the in vitro diagnostics process of the patient sample, in some examples. This verification can include limiting the analysis to a test within multiple test options of the patient sample that are available in the reader 1202 based on the cartridge verification.

A positive engagement or lock in the reader 1202 can engage the cartridge 1204 when properly and fully inserted. This engagement can also provide a tactile, audible, or visual cue to the user to signify proper insertion or interfacing of the cartridge 1204 and reader 1202. An example positive engagement or lock can include a notch and protrusion arrangement, the notch is sized to receive and releasably restrain the protrusion when engaged such that the notch of one element, the reader 1202 or cartridge 1204, engages the protrusion on the opposite element, reader 1202 or cartridge 1204, to releasably connect, interface with or engage the two elements, the reader 1202 and cartridge 1204, together. When prompted, such as when the analysis is completed or in a cartridge error situation, the user can be alerted to remove the cartridge 1204 from the reader 1202.

Also, the reader 1202 includes processing circuitry 1228 that has multiple modules that prepare the patient sample, run the test on the sample, and analyze the results. The processing circuitry 1228 has an electrophoresis module 1230 that can run the test, analyze the results, and optionally prepare the sample for the test. The processing circuitry can alter the processing of the sample analysis data based on the type of cartridge 1204 inserted within the reader 1202.

Insertion of the cartridge 1204 into the cartridge interface 1222 of the reader 1202 can automatically initiate or prompt a user to initiate analysis of the patient sample contained within the cartridge 1204. An actuator or sensor can be connected to the processing circuitry 1228 of the reader 1202 and triggered by or sense the insertion of the cartridge 1204 to automatically initiate or to prompt a user to initiate the analysis of the patient sample. Initiating analysis of the patient sample can include powering-up, preparing, or running the various analyses systems or devices, such as an electrophoresis diagnostic test, for example. In some examples, the user need only insert the cartridge 1204 in the reader 1202 to actuate or trigger the entire diagnostics process to an output.

The reader 1202 can include a single cartridge interface 1222 or can include multiple cartridge interfaces in the same reader. The example readers with multiple cartridge interfaces allow the reader to analyze multiple patient samples simultaneously or in succession by allowing more than one cartridge 1204 to interface with the reader 1202. Additionally, each of the multiple cartridge interfaces can accept the same or different cartridges to perform the same or different tests, respectively. Further, in conjunction with a multi-or single cartridge interface, a guide, rack, carousel or system can hold multiple cartridges in preparation for analysis. The guide, rack, carousel or system can feed or guide, actively or passively, cartridges to the reader by the cartridge interface allowing multiple patient samples or cartridges to be analyzed with minimal interruption between the analyses.

The electrophoresis module 1230 of the processing circuitry 1228 in the reader 1202 shown in FIG. 12 initiates the electrophoresis test. The electrophoresis module 1230, alone or in conjunction with the processing circuitry 1228, can control the electrophoresis test, including electric potential application time or level. The electrophoresis module 1230 can supply electrical power from a power source 1232 of the reader 1202 to the cartridge 1204, or electrophoresis substrate directly, to establish the necessary electric potential across the electrophoresis substrate for testing.

The band analysis module 1234 receives captured image(s) from an optical imager 1236 and analyzes or evaluates the images or electrophoresis test results or any other band detection characteristic(s) related to or otherwise based on the electrophoresis test results. The optical imager 1236 can include an imaging device, such as a digital image sensor, to capture an image of the electrophoresis substrate and the banding thereon during or at the conclusion of the electrophoresis test. Using the captured image data, each of the bands can be associated with one or more compounds or components of the patient sample and optionally the concentration of one or more components associated with a band can be determined for quantifying the target compound or biomarker concentration. The band analysis module 1234 receives captured image(s) from the optical imager 1236 for analysis including images captured during the active assay 1238 and at the end of the active assay run when the electric potential is removed. Individually or collectively, the captured image(s) both during the active assay and at the end of the assay run create a migration pattern 1240. The migration pattern 1240 is based on the migration of the components in the patient same as they move across the substrate during and at the end of the assay when the electric potential is removed. While optional, the active assay imaging 1238 is very helpful to show migration of the bound complex and other patient sample components as they migrate across the substrate, which helps create the migration pattern 1240 for the test. The migration pattern 1240 and optionally a migration velocity 1242 is used by the band analysis module 1234 to compare bands associated with different components of the patient sample during and after the assay run.

The reader 1202 can also include an optional sample treatment chamber 1244 or module that controls treatment of the patient sample within the reader 1202. The sample treatment 1244 can include a buffer solution for use with the electrophoresis substrate, markers to add to the patient sample, diluents or other solutions or compounds for use in the electrophoresis testing. The sample treatment(s) 1244 can be contained within removable cartridges to ease replacement or change of the sample treatment 1244. Alternatively, the reader 1202 can include internal containers for storing the sample treatment 1244. Associated tubing, systems or components can be included to facilitate the transfer of the sample treatment 1244 to the cartridge 1204 or other systems or components of the reader 1202.

The positioning and structure of the cartridge 1204 within the reader 1202 can be such that it properly aligns with test components controlled by the electrophoresis module 1230 when inserted into the reader 1202. For example, the cartridge 1204 is inserted to that the electrodes on the substrate come into electrical contact with an electric potential source and the substrate 1218 is visible by the optical imager 1236 for image capture during and after the test.

The reader 1202 includes a light source 1227, as discussed above. The light source 1227 can be the same light source that excites the fluors 812, if they are present, but emits light, such as white light, to help illuminate the electrophoresis substrate 1218 and assist with capturing image(s) for the electrophoresis results. The light source 1227 could be used for multiple reasons with the correct filter placed to target the wavelength of the desired light transmission—whether it is employed for fluorescence, image capture, or both. The light emitted by the light source 1227 can have constant or varying properties, such as a wavelength and intensity or a frequency of the emitted light. The light source 1227 can include one or more illumination elements to generate light having the required or desired properties to assist with imaging or analyzing the electrophoresis results.

The reader 1202 can include an internal power source 1232 that supplies the necessary power to run the components, elements or systems of the reader to perform analysis of patient samples or preserve a minimal, required functionality of the reader 1202. The power source 1232 can supply power to the processing circuitry, the electrophoresis module, the electrophoresis band detection module or other component, elements or systems of the reader. The power source 1232 can include one or more batteries or other energy storage devices that provide a required or desired level of power for the reader 1202. Additionally, the power source 1232 or a portion thereof can be external to the reader 1202 and connected thereto as needed or required. External power sources can include batteries or other energy storage devices or a connection to a nearby power source such as a generator, municipal power, or solar array.

The reader 1202 can include an output 1246 that includes one or more audio 1248, visual 1250, tactile 1252 or other outputs. In other examples, the output 1246 is data and does not include audio, visual, or tactile outputs. The output 1246 shown in FIG. 12 communicates information regarding the status of the reader 1202, the results of analysis of a patient sample, instructions regarding use of the reader 1202 or other information to a user or other computing device. For example, the output 1246 can output data including the capture image(s) of the active assay or interpretative data that indicates presence or absence of a disorder, condition, infection, or disease in the patient sample. An example can include the identification of and quantification or partial quantification of bound complex in the patient sample.

The reader 1202 can also include temperature control (not shown in FIG. 12). The temperature control can actively or passively control the temperature of at least a portion of the reader. Active temperature control can include heating or cooling a portion of the reader 1202 or cartridge 1204. Temperature control can also include heating one portion of the reader 1202 or cartridge 1204 and cooling another portion of the reader 1202 or cartridge 1204. The temperature control can include a refrigeration system, resistive heater, infrared heater, thermoelectric elements, radiator, or other temperature control devices or systems. Passive temperature control can include structures to contain a thermal material in portions of the reader 1202 or cartridge 1204. This can include containers or supporting structures for ice, hot water, ice packs, and other thermal materials. The holders retain the thermal material in portions of or about components, elements or systems of the reader 1202 or cartridge 1204.

The reader 1202 or cartridge 1202 can also include a filter. For example, the filter can remove cells from a patient sample, such as blood cells from a blood sample, which results in a patient sample of plasma or serum without blood cells. The filter can attract, extract, collect or otherwise remove unwanted components or particles in a patient sample of the cartridge 804 or concentrate the desired components or particles. Filtering the patient sample can occur as the patient sample is transferred from the cartridge 1204 into the reader 1202 or the patient sample can be transferred from the cartridge 1204, through the filter and back into the cartridge 1204 for analysis or it can be internal to the cartridge 1204. The filter can include structural and chemical features that allow the filter to remove desired or required components from the patient sample. The filter can be affixed in a stationary position to contact the patient sample or can moveable through the patient sample to filter the patient sample.

Processing circuitry 1228 can be included in the reader to receive input from various components, elements or systems, such as the electrophoresis module 1230 of the reader 1202. The processing circuitry 1228 can process the received inputs to perform analysis of the patient sample and output results or data of that analysis. The processing circuitry 1228 can initiate or control the analysis of a patient sample within a cartridge 1204. The processing circuitry 1228 can include preset routines, which may be defaults or selectable by the user, which can be executed by the reader to analyze a patient sample. The preset routines can include prompts for user input received from a user interface 1254 or input from another computing device or the processing circuitry can prompt a user for input before, during, or after analysis of a patient sample. User prompts can include acknowledgement or authorization to proceed through one or more portions of the analysis process. Alternatively, the processing circuity 1228 can initiate, perform, or direct the analysis of the patient sample automatically without user prompts. The processing circuitry 1228 can proceed through the various processes and procedures of an analysis of a patient sample, engaging any one or more of the reader 1202, remote or near transmission system or computing devices, and collecting the analysis data. The processing circuitry 1228 can further automatically process the collected data and transmit a result to a user or other, including an indication the analysis is complete, information regarding the analysis or other indications. The processing circuitry 1228 can also transmit the collected data to an external system or device for processing and can transmit a result to the user or the result can be transmitted by one or more of an external system, computing device, or server, such as a user device 1256 or a remote computing device, server, or network 1258.

The reader 1202 can communicate with other remote computing device(s), server(s), or network(s) 1258 through data transmitted and received from a transmitter 1260 or receiver 1262, respectively, which in this example are integrated in a transceiver. The data transmission can occur through a physical connection or input 1264, such as a local area network (LAN), Universal Serial Bus (USB), or a wireless connection 1266, such as Bluetooth®, for example. In an example, the reader 1202 can communicate with a remote computing device, server, or network 1258 over a network 1268, which allows the reader 1202 to upload patient sample analysis to a patient's health records stored on the remote server, for example. The reader 1202 can transmit or receive communication to and from the reader 1202 and another device or system 1256, 1258. In another example, information on the patient can be downloaded to the reader 1202 and output to a display integrated in the user interface 1254, for example, or output or used in the analysis(es). Additionally, a local user device 1256 can be connected to the reader 1202 for user control or interaction with the reader 1202 during or after the assay is run. For example, a user tablet or laptop 1256 can be connected to the reader 1202 by short range communication, which allows the user tablet 1256 to receive test data and control the data analysis and results.

The reader 1202 can include an internal memory 1270 that has various data stores including an optional empirical data store 1272 and a patient health records data store 1274, by way of example as shown in FIG. 12. Other data can be stored in the reader's memory 1270, as needed. The empirical data store 1272 can receive data about other assays including band analysis data that helps identify and quantify detected bound complex 1216, for example. Empirical data known or previously captured as being associated with a known component can be used as a guide to evaluate whether a band is associated with a that component in the patient sample. It can also serve as a way to store data on the tests being run, determine trends, and export the data to remote computers, servers, or networks 1258 for further analysis. The memory 1270 can also include a patient health record data store 1274 that includes information about a particular patient that is stored internally or transmitted to a remote computer, server, or network 1258.

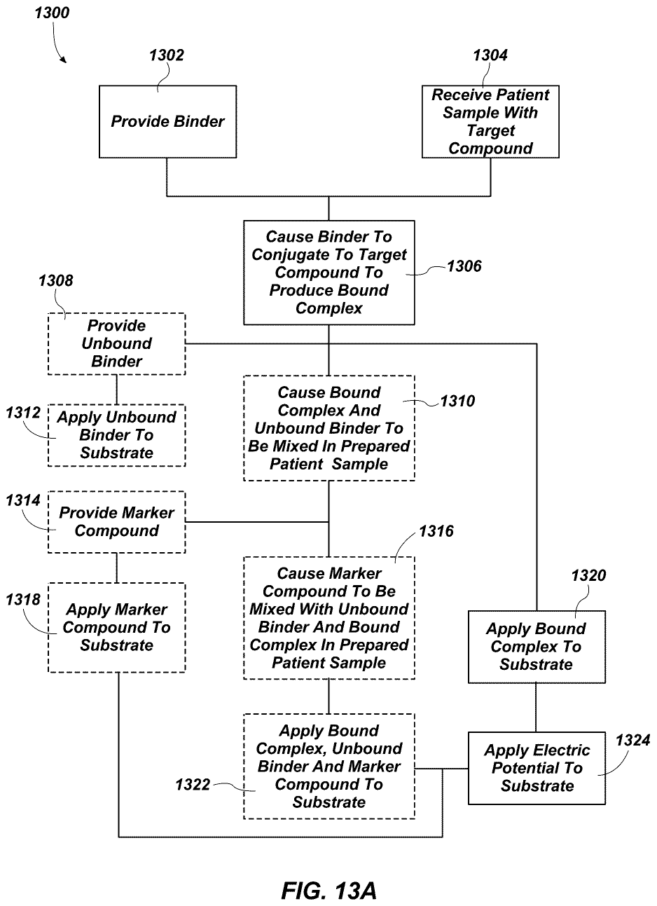

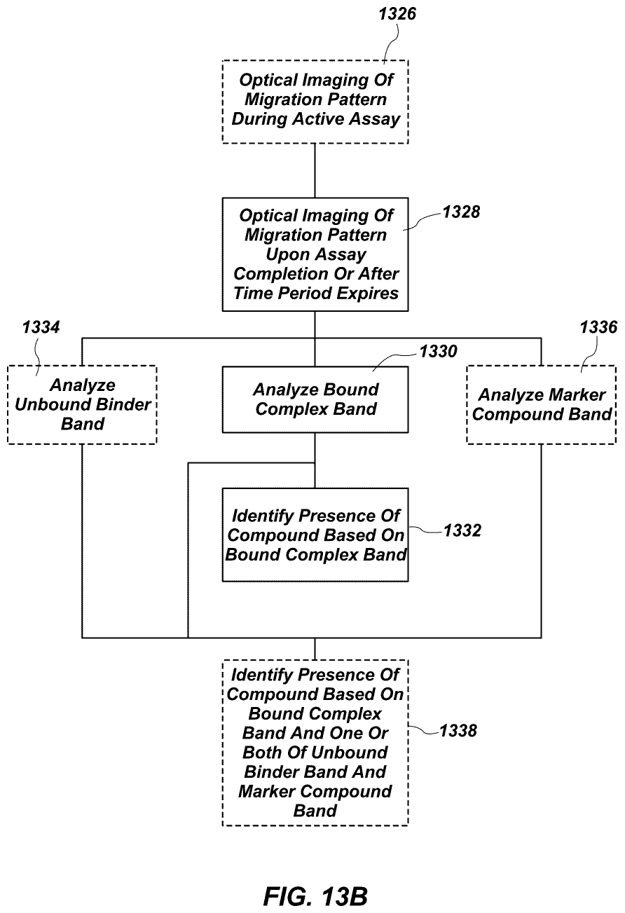

FIGS. 13A and 13B show steps in a method of identifying presence of a target compound or biomarker in a patient sample 1300. The binder is provided 1302 and a patient sample with a compound is received 1304. The binder is caused to conjugate with the target compound to produce a bound complex 1306, which can be accomplished by incubating the binder and target compound for a period of time, for example. In an example, unbound binder is provided 1308 and the bound complex and unbound binder that does not conjugate with target compound is mixed in the prepared patient sample 1310. Alternatively, the unbound binder, as discussed above, can be directly applied to the substrate without mixing with the prepared patient sample 1312. Further, marker compound, as discussed above, can be provided 1314 and the marker compound can be caused to be mixed with the prepared patient sample 1316 or the marker compound can be directly applied to the substrate 1318. If no unbound binder is provided 1308 and no marker compound is provided 1314, then the bound complex is applied to the substrate 1320. However, if the unbound binder and marker compound are both provided, then both of them along with the bound complex are applied to the substrate 1322. After the substrate has the prepared patient sample, the electric potential is applied 1324.

The applied electric potential 1324 produces a migration pattern on the substrate. The migration pattern is optically imaged during the active assay 1326 and after the assay is completed or after a predetermined period of time 1328. As discussed above, the migration pattern produces bands of molecules having like charge and mass. Those bands are analyzed, and specifically in the example with a patient sample that includes the target compound, the bound complex band is analyzed 1330. If no unbound binder or marker compound are present on the substrate, then the presence of the compound is determined based on the bound complex band 1332. If unbound binder is present on the substrate, it produces an unbound binder band that is also analyzed 1334. Likewise, if marker compound is present on the substrate, it produces a marker compound band that is analyzed 1336. If unbound binder and marker compound are present, then presence of the target compound is identified based on the bound complex band and one or both of the unbound binder band and the marker compound band.

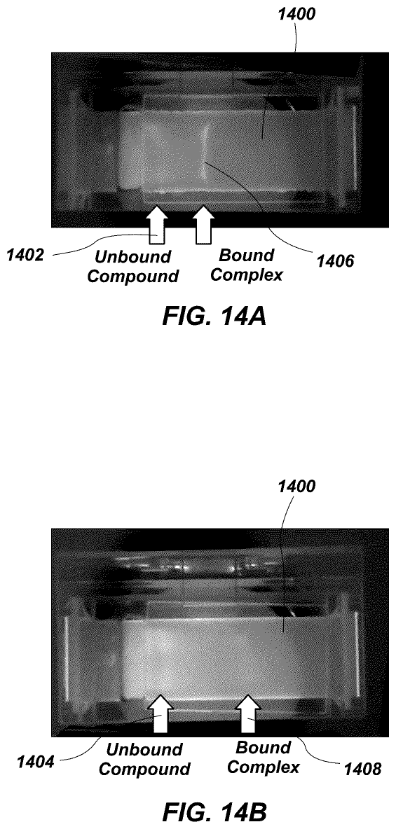

FIGS. 14A and 14B show migration patterns of substrates for two positive samples—both having target compound—but each has a different concentration of the target compound 1400. The different concentrations of the bands correlate to different intensity of the respective optically detectable bands. When these bands are optically imaged, the unbound compound band 1402 appears the same or relatively similar in the two tests with different concentrations of the bound complex. FIG. 14A shows an unbound compound band 1402 with relatively similar band characteristics like migration velocity, pixel intensity, and width to the unbound compound band 1404 in FIG. 14B. The unbound compound bands 1402 and 1404 have similar band characteristics because their controlled charge and mass produces a similar migration pattern. The bound complex band 1406 in FIG. 14A have a higher concentration than the bound complex band 1408 in FIG. 14B. These bands 1406, 1408 differ substantially in pixel intensity and width.

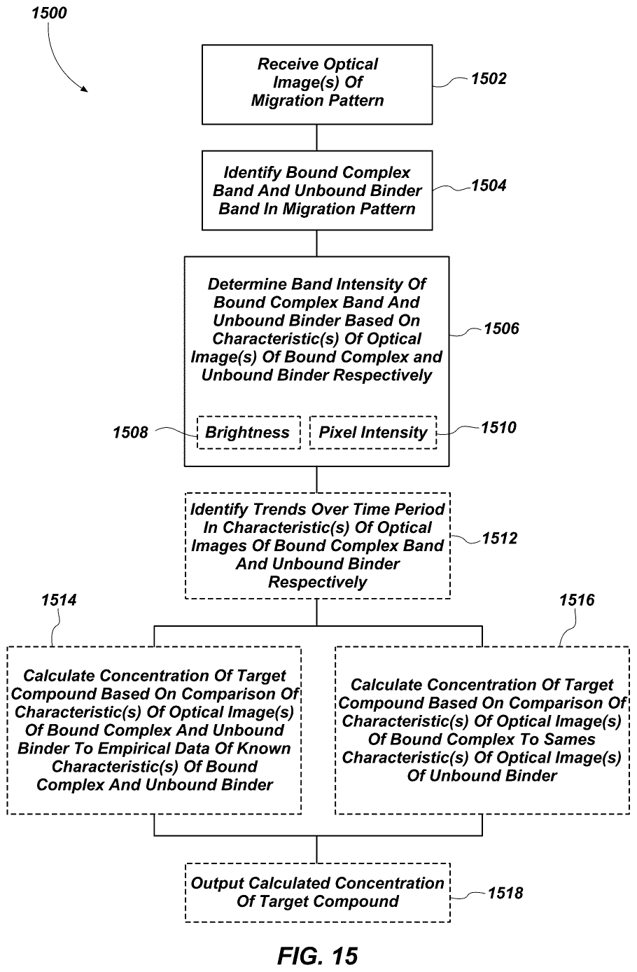

FIG. 15 shows an example method of analyzing band intensity of unbound binder and bound complex in a patient sample 1500. This band intensity analysis can determine quantitative results on the concentration of the target compound in the patient sample. The processor can receive optical images of a migration pattern 1502. The migration pattern includes multiple bands. As discussed above, a bound complex band can be identified and, in this example shown in FIG. 15, an unbound binder band is also identified 1504. An intensity of the bound complex band and the unbound binder are determined based on characteristics of the received optical images for the bound complex and the unbound binder, respectively 1506. The characteristics can include brightness 1508 of each band and the pixel intensity 1510 of each band. In some examples, trends in these characteristics are identified over a period of time 1512. The concentration of the target compound is based on analysis of the characteristics of the band intensity for each band.

In one example, the concentration of the target compound is calculated based on a comparison of the characteristics of the optical images—such as brightness or pixel intensity—to empirical data known to have discrete levels of target compound concentration in the bound complex band and unbound binder in the unbound binder band 1514. In other examples, the concentration of the target compound is calculated based on a relative comparison of the characteristics of the optical images—the brightness or pixel intensity—between the bands 1516. This means that the sensed brightness or pixel intensity of the bound complex is compared to the brightness or pixel intensity of the unbound binder having a known concentration of target compound. The unbound binder differential or relative comparison correlates to the concentration of the target compound in the bound complex. The calculated concentration of the target compound in then output 1518 on an output, such as a user interface of the reader, or transmitted to a user device or remote computer, server, or network, in some examples.

Though certain elements, aspects, components or the like are described in relation to one embodiment or example, such as an example diagnostic system or method, those elements, aspects, components or the like can be including with any other diagnostic system or method, such as when it desirous or advantageous to do so.

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific embodiments are presented by way of examples for purposes of illustration and description. They are not intended to be exhaustive of or to limit this disclosure to the precise forms described. Many modifications and variations are possible in view of the above teachings. The embodiments are shown and described in order to best explain the principles of this disclosure and practical applications, to thereby enable others skilled in the art to best utilize this disclosure and various embodiments with various modifications as are suited to the particular use contemplated. It is intended that the scope of this disclosure be defined by the following claims and their equivalents.

What is claimed is:

1. A diagnostic system, comprising:
a processor configured to:
receive data including:
detection of a bound hemoglobin A1c (HbA1c ) complex in a patient sample, the bound HbA1c complex including HbA1c conjugated with a binder having a binder profile, the binder profile having one or both of a controlled binder charge and a controlled binder mass;
a migration pattern of the bound HbA1c complex produced in response to an applied electric potential to a substrate on which the bound HbA1c complex is applied, the migration pattern generated as the bound HbA1c complex migrates across the substrate at a migration velocity over an interval to produce a bound HbA1c complex band on the substrate;
determine presence of HbA1c in the patient sample based on the bound HbA1c complex band and one or both of the migration pattern and a feature or characteristic of the migration velocity; and
an output configured to output data that includes the determination of the presence of the HbA1c.

2. The system of claim 1, wherein the binder has a controlled binder charge that changes a charge of unconjugated HbA1c to a total net charge of the bound HbA1c complex.

3. The system of claim 1, wherein the binder has a controlled binder mass that increases a mass of unconjugated HbA1c to a total net mass of the bound HbA1c complex.

4. The system of claim 1, wherein the binder has a controlled binder charge that increases a charge of unconjugated HbA1c to a total net charge of the bound HbA1c complex and a controlled binder mass that increases a mass of unconjugated HbA1c to a total mass of the bound HbA1c complex.

5. The system of claim 1, wherein the binder has a controlled binder charge of zero that does not change a total net charge of the bound HbA1c complex and a controlled binder mass that increases a mass of unconjugated HbA1c to a total mass of the bound HbA1c complex.

6. The system of claim 1, wherein the data with the migration pattern of the bound HbA1c complex further includes:
data relating to a single bound HbA1c complex band correlating to a single HbA1c charge state of the bound HbA1c complex; and
an identification of presence of the HbA1c based on the single bound HbA1c complex band and one or both of the migration pattern and the feature or characteristic of the migration velocity.

7. The system of claim 1, wherein the data signal with the migration pattern of the bound HbA1c complex further includes:
data relating to a respective bound complex band for each of respective multiple HbA1c charge states of the bound HbA1c complex; and
an identification of presence of the HbA1c based on the multiple respective bound complex bands and one or both of the migration pattern and the feature or characteristic of the migration velocity.

8. The system of claim 1, wherein the migration pattern includes two steps, a first step including a total hemoglobin (Hbₜₒₜₐₗ) band that separates from a marker compound and a second step including an HbA1c band that separates from the Hbₜₒₜₐₗ band.

9. The system of claim 8, wherein the second step further includes a hemoglobin A2 (HbA2) band that separates from the Hbₜₒₜₐₗ band.

10. The system of claim 8, wherein the second step further includes one or more of a hemoglobin A0 (HbA0) band, a hemoglobin A2 (HbA2) band, a hemoglobin S (HbS) band, a hemoglobin C (HbC) band, and a hemoglobin E (HbE) band that separates from the Hbₜₒₜₐₗ band.

11. The system of claim 1, wherein the binder further includes a label that binds to one or both of the binder or the compound to create a labeled bound binder-target complex, and wherein the processor is further configured to:
generate the migration pattern of the labeled bound complex band based on the labeled bound binder-target complex migrating across the substrate at the migration velocity over the interval, the migration pattern producing the labeled bound complex band.

12. The system of claim 11, wherein the processor is further configured to:
receive data relating to a fluorescence emission of the labeled bound binder-target complex, the fluorescence emission produced by the fluorescence label when an excitation energy excites the fluorescence label and causes the fluorescence label to fluoresce;
identify presence of the compound based on the fluorescence emission, the labeled bound complex band, and one or both of the migration pattern and the feature or characteristic of the migration velocity.

13. The system of claim 12, wherein the excitation energy has a wavelength that causes the fluorescence emission of the labeled bound binder-target complex.

14. The system of claim 12, wherein the wavelength causes a fluorescence emission of one of the binder, the compound, or both the binder and the compound.

15. The system of claim 12, wherein the excitation energy includes multiple wavelengths, at least one of the wavelengths causing the fluorescence emission of the labeled bound binder-target complex.

16. The system of claim 15, wherein a second wavelength of the excitation energy causes fluorescence emission of a second compound or a second binder bound to the second compound, a second label bound to the second compound or to the second binder, or one or more of the second compound, the second binder, and the second label in the patient sample.

17. The system of claim 16, wherein the processor is further configured to identify presence of the second compound, the second binder bound to the second compound, the second label, or multiple or all of the second compound, the second binder, and the second label based on the fluorescence emission of the second compound, the second binder, the second label, or multiple or all of the second compound, the second binder, and the second label.

18. The system of claim 1, wherein the processor is further configured to:
receive data relating to a fluorescence emission of the bound HbA1c complex, the fluorescence emission produced by the binder, the HbA1c, or the binder and the HbA1c when an excitation energy excites the bound HbA1c complex and causes the bound HbA1c complex to fluoresce;
identify presence of the HbA1c based on the fluorescence emission, the bound HbA1c complex band, and one or both of the migration pattern and the feature or characteristic of the migration velocity.

19. The system of claim 1, wherein the processor is further configured to:
receive data including an HbA1c charge state and an HbA1c mass of the HbA1c; and
generate the migration pattern of the bound HbA1c complex based on the HbA1c charge state, the HbA1c mass, and the one or both of the controlled binder charge and the controlled binder mass.

20. The system of claim 1, wherein the processor is further configured to identify the presence of the HbA1c based on the bound HbA1c complex band and the migration pattern.

21. The system of claim 1, wherein the processor is further configured to identify the presence of the HbA1c based on the bound HbA1c complex band and the feature or characteristic of the migration velocity.

22. The system of claim 1, wherein the processor is further configured to identify the presence of the HBA1c based on the bound HBA1c complex band, the migration pattern, and the feature or characteristic of the migration velocity.

23. The system of claim 1, wherein the processor is further configured to:
determine a band concentration, band intensity, band density, or band spatial intensity of the bound HbA1c complex band based on an optical characteristic or an optical feature of the bound HbA1c complex band; and
determine a relative concentration, quantification, density, or band spatial intensity of the HbA1c in the patient sample based on the band concentration, band intensity, or band density, respectively, of the bound HbA1c complex band and a concentration, quantification, or density of another type of Hb band.

24. The system of claim 23, wherein the output is further configured to output the data signal to include the determination of the presence of the HbA1c and the concentration, quantification, or density of the HbA1c.

25. The system of claim 23, wherein the processor is further configured to:
determine the band concentration, band intensity, or band density based on a characteristic or feature of a video, an optical image, or a series of optical images of the bound HbA1c complex band at one or more times in the migration pattern; and
determine a concentration, quantification, or density of the HbA1c based on the band concentration, band intensity, or band density, respectively, of the bound HbA1c complex band.

26. The system of claim 23, wherein the processor is further configured to determine a diagnostic category of the HbA1c complex band based on the concentration, quantification, or density of the bound HbA1c complex band, and wherein the output is further configured to output the diagnostic category of the HbA1c complex band.

27. The system of claim 1, wherein the processor is further configured to determine a diagnostic category of the HbA1c complex band based on the presence of HbA1c in the patient sample, and wherein the output is further configured to output the diagnostic category of the HbA1c complex band.

28. The system of claim 27, wherein the diagnostic category includes healthy, prediabetes, and diabetes categories for the bound HbA1c complex band.

29. The system of claim 27, wherein the processor is further configured to:
receive a data signal that includes a migration pattern of an unbound binder based on the controlled charge state, the controlled mass, or both the controlled charge state and the controlled mass, the migration pattern of the unbound binder generated as the unbound binder migrates across the substrate at a migration velocity over the interval to produce an unbound binder band on the substrate;
determine a band intensity of the unbound binder based on a characteristic of the unbound binder band;
compare the band intensity of the unbound binder band and the band intensity of the bound complex band; and
determine a concentration or quantification of the HbA1c based on the comparison of the band intensity of the bound complex band and the band intensity of the unbound binder band.

30. The system of claim 29, wherein the processor is further comprised to:
receive a data signal with an optical image of the bound HbA1c complex band and an optical image of the unbound binder band during migration of the bound complex and the unbound binder, respectively; and
identify the presence of the HbA1c based on a feature of the optical image of the bound HbA1c complex band and the optical image of the unbound binder band.

31. The system of claim 30, wherein the processor is further configured to transmit an instruction to capture the optical image of the bound complex band and the optical image of the unbound complex band.

32. The system of claim 31, wherein the processor is further configured to:
transmit an instruction to capture multiple optical images of the bound HbA1c complex band and multiple optical images of the unbound binder band; and
receive the data signal with the migration pattern of the bound HbA1c complex that includes features or characteristics of the multiple optical images of the bound HbA1c complex band and features or characteristics of the multiple optical images of the unbound binder band;
identify the presence of the HbA1c based on the one or both of the features or characteristics of the multiple optical images of the bound HbA1c complex band and the features or characteristics of the unbound binder band and one or both of:
the migration pattern of the bound HbA1c complex,
the migration pattern of the unbound binder, and
a feature or characteristic of the migration velocity.

33. The system of claim 1, wherein the processor is further configured to:
receive multiple or continuous optical images of the migration pattern of the bound HbA1c complex band over the interval; and
identify the presence of the HbA1c based on the bound HbA1c complex band, the one or both of the migration pattern and the feature or characteristic of the migration velocity, and the multiple or continuous optical images of the migration pattern.

34. The system of claim 1, wherein the processor is further comprised to:
in response to the applied electric potential, generate the migration pattern of the bound HbA1c complex and a migration pattern of a marker compound, the marker compound having a marker concentration, the migration pattern of the marker compound generated as the marker compound migrates across the substrate at a marker migration velocity over the period of time to produce a marker compound band.

35. The system of claim 34, wherein the processor is further configured to identify the presence of the HbA1c based on comparing the bound HbA1c complex band to the marker compound band.

36. The system of claim 1, wherein the substrate is photobleached electrophoresis paper, and wherein:
the processor is further configured to receive a data signal with an optical image of the bound complex band; and
the photobleached electrophoresis paper is photobleached before the optical image is captured.
